(19) United States
(12) Patent Application Publication
(10) Pub. No.: US 2016/0339146 A1
(43) Pub. Date: Nov. 24, 2016

(54) Coating Method and Materials

(71) Applicant: Gregory Cauchon, Thousand Oaks, CA (US)

(72) Inventor: Gregory Cauchon, Thousand Oaks, CA (US)

(21) Appl. No.: 14/995,778

(22) Filed: Jan. 14, 2016

Related U.S. Application Data
(62) Division of application No. 14/720,783, filed on May 23, 2015.

(51) Int. Cl. A61L 27/28; A61L 27/20

(52) U.S. Cl. CPC A61L 27/28; A61L 27/20

(57) ABSTRACT

A system consisting of a component coating a particulate scaffold is able to transfer the coating to an exposed surface in order to yield beneficial effects. The system is useful for many applications, including the treatment of individuals suffering from a disease, cancer, an ailment, an infection, a toxin, or other syndrome, as well as for numerous non-treatment purposes requiring the coating of exposed surfaces.

This application is a divisional of U.S. patent application Ser. No. 14/720,783, filed May 23, 2015, which claims the benefit of priority pursuant to 35 U.S.C. §119(e) to U.S. Provisional application 62/002,355, filed on May 23, 2014, which is hereby incorporated by reference in its entirety.

### BACKGROUND

The coating of surfaces, including everything from molecular and planar surfaces to complex macroscopic 3-D objects such as prosthetic devices, is of great economic and medical importance. In particular, the coating of microscopic objects with useful materials is an increasingly important family of industrial processes which will be needed to meet the coming challenges in the fields of microelectronics, biotechnology, nanotechnology, and cleantech, among others. New coating technologies are therefore greatly needed and are continuously being sought in a variety of industries, and any improvement in such a process which can help reduce manufacturing costs or enable product availability or improve product quality can provide important competitive advantages.

Microorganisms such as bacteria, viruses, molds, and fungi have plagued higher organisms such as humans and other mammals throughout history. Only with the discovery of vaccination in the twentieth century did mankind begin to learn to harness the power of the naturally-occurring adaptive immune response to fight off infections. But only certain immunogens have thus far proven amenable to this approach; while many immunogens have proven useful for therapeutically inducing immunity in humans and animals, others have failed to produce long-lasting immunity, and many have produced no immunity at all. For example, there are now safe and effective vaccinations for diseases such as pertussis, whooping cough, measles, and chicken pox. But other diseases remain beyond the reach of vaccines, including HIV, SARS, most cancers, and even some forms of influenza. In animals, new diseases are still being discovered regularly, and vaccines against them are of increasing economic importance. And in plants, there are essentially no commercially-available vaccines. Thus, new and improved vaccination methods are continuously being sought to meet new and existing challenges.

Active immunization is the process of stimulating the production of antibodies by the introduction of a specific antigenic toxin into an animal. In order to elicit an immune response, the toxin must be strong enough to stimulate the immune system but not so strong as to kill the host animal. Many relatively weak microorganisms can be used intact, either live or killed, while others have to be attenuated further in order for the host organism to survive long enough to produce antibodies. A toxin which is inactivated in this manner is called a toxoid, and there are various reagents which are commonly used for producing toxoids for use as vaccines, including, for example, formaldehyde and tannic acid. Unfortunately, even vaccines produced in this manner can exhibit serious side effects, including swelling, pain, fever, vomiting, shock, brain damage, and even death. Thus, there is a great need for new methods and materials which allow the use of dangerous and highly toxic microorganisms and their components as well as other materials as toxoids for inducing immunity.

Plants also possess immune systems, although they lack the acquired (also referred to as ‘adaptive’) immune system of vertebrates, with its mobile cellular and humoral components. The plant immune system can be thought of as consisting of at least three branches, one of which uses transmembrane pattern-recognition receptors (PRRs) to detect and bind to slowly-evolving microbe-associated or pathogen-associated molecular patterns (MAMPS or PAMPS) such as flagellin or bacterial lipopolysaccharide (LPS). Another branch of plant immunity makes use of polymorphic nucleotide-binding or leucine-rich repeat (NB-LRR) proteins, with this capability confined largely to the insides of cells. There is also emerging evidence for a strong and adaptive RNA-based component forming a third branch of plant immunity. These recent developments suggest that it will be possible to generate immune responses in plants using a wide range of interesting and valuable antigens as the technology develops. Thus, potential plant antigens and new methodologies are of great value in many areas of agriculture, forestry, and oceanography, such as in the production of food, fibers, lumber, and biofuels, as well as any field in which the induction of plant immunity would be of value.

The ability of certain components of grapes and wine to bind to and inhibit microorganisms such as viruses and bacteria has been known since the pioneering work of Jack Konowalchuk in the 1970s. It was suggested that bactericidal effects might be common to all polyphenols, including those that are a component of grapes, and this property was presumed to result from their protein-binding capability. Grape skins and wines are rich sources of polyphenols, but the interactions of these naturally-occurring materials with microorganisms have generally proven weak and difficult to control, while the mechanism(s) behind their activity remains obscure, so that thus far no therapeutic or other commercial products have resulted from these observations of anti-microbial efficacy.

Tannins are monomers and oligomers of a number of flavonoid components, including catechin and epicatechin, gallocatechins, galloepicatechins, flavanols, flavonols, flavandiols, leucocyanidins, and/or anthocyanidins. Tannins have long been used to attenuate toxins in addition to their larger application for purposes such as the tanning of leather. For example, the use of tannins from the persimmon fruit, genus Diospyros, was described in U.S. Pat. No. 4,172,126. In fact, a model test for the ability of tannins to inactivate toxins and their side effects has been to observe the effects of the action of tannins on snake venoms. In this process, an effective amount of tannins are contacted with the microbial antigen to attenuate the vaccine or inactivate the microbial toxin, producing a toxoid which is more suitable for use in generating an immune response without killing or seriously injuring the host.

Proanthocyanidins are a type of condensed, or polymerized, tannins which consist of linear chains of the flavan-3-ols catechin and epicatechin. They are called proanthocyanidins because they can be converted to anthocyanidins when they are depolymerized under oxidative conditions. Because these monomers possess different structures, a wide variety of polymeric proanthocyanidins are possible. Large polymers of these monomers are the predominant proanthocyanidin species in most plants, with average molecular masses above 2,000 Daltons, and these have been reported to possess protein-binding capability and possibly a biological role. For example, a procyanidin monomer has been shown to exhibit antiviral activity against the Herpes simplex virus.

The present invention relates especially to the fields of human and veterinary medicine, microbiology, and virology, but also has broader applicability to industries ranging from biotech and pharmaceuticals to microelectronics, nanomaterials, packaging, and consumer products, as well as agriculture, forestry, oceanography, and the production of food, fibers, lumber, and biofuels. The present invention also applies to any field in which the efficient or precise coating of particles or surfaces is useful or desireable, including, without limitation, the use of such a coated particle to cover a macroscopic or microscopic object to be used to treat a disease or condition of a living organism or an individual, or else to be used directly in the treatment of a living organism or individual in need of such treatment.

### SUMMARY

Aspects of the present specification disclose a composition comprising a scaffold and a component, wherein the component coats a surface of the scaffold. Aspects of the present specification include a component-coated composition comprising a scaffold and one or more components, wherein the one or more component coats a surface of the scaffold. A component disclosed herein includes a tannin, a pseudotannin and/or a proanthocyanidin. A scaffold disclosed herein may comprise a biological organism, a viral particle, a viron, a protein, a protein cage, a peptide, an antibody, a venom, a toxin, a vault, a nucleic acid, a fat, a lipid, a micelle, a liposome, a carbohydrate, a therapeutic compound, an active ingredient, a polymer latex, a metal object, a composite object, a fiber, a bead, and/or a rubber object.

Aspects of the present specification disclose a method for coating a surface of a target particle. Aspects of the present specification include a method comprising a) producing, dissolving, or suspending a scaffold in a suitable solvent; b) mixing or loading one or more components onto the dissolved or suspended scaffold to form a component-coated scaffold; c) exposing the component-coated scaffold to a surface of a target particle; and d) transferring at least some of the one or more components onto the surface of the target particle. Other aspects of the present specification include a method comprising for coating a surface of a target particle, the method comprising: a) producing, dissolving, or suspending a scaffold and one or more components in a suitable solvent; b) exposing the component-coated scaffold to a surface of a target particle; and c) transferring at least some of the one or more components onto the surface of the target particle. Aspects of the present specification include a method comprising a) producing, dissolving, or suspending one or more components to form a self-assembled scaffold, wherein the one or more components includes one or more tannins; b) exposing the self-assembled scaffold to a surface of a target particle; and c) transferring at least some of the one or more components onto the surface of the target particle. A component disclosed herein includes a tannin, a pseudotannin and/or a proanthocyanidin. A scaffold disclosed herein may comprise a biological organism, a viral particle, a viron, a protein, a protein cage, a peptide, an antibody, a venom, a toxin, a vault, a nucleic acid, a fat, a lipid, a micelle, a liposome, a carbohydrate, a therapeutic compound, an active ingredient, a polymer latex, a metal object, a composite object, a fiber, a bead, and/or a rubber object. A target particle may be a microorganism, a cell, a subcellular organelle, or a synthetic object. A microorganism includes a virus, a bacterium, a mold, a fungi, or a protozoan. A synthetic object comprises an outer and/or an inner surface of a prosthetic device or a surface on a diagnostic test plate.

Aspects of the present specification disclose a method of producing an immune response an individual. Aspects of the present specification include a method comprising administering a pharmaceutical composition comprising a composition comprising a scaffold and a component disclosed herein.

Aspects of the present specification disclose a method treating an individual suffering from a disease, an infection, a cancer, a skin ailment, or other syndrome. Aspects of the present specification include a method comprising administering a pharmaceutical composition comprising a composition comprising a scaffold and a component disclosed herein. A pharmaceutical composition may comprises one or more component-coated microorganisms. The one or more component-coated microorganism may be a proanthocyanidin-coated microorganism like, e.g., a proanthocyanidin-coated virus, a proanthocyanidin-coated toxoid, a proanthocyanidin-coated bacterium, a proanthocyanidin-coated mold, a proanthocyanidin-coated fungus, and/or a proanthocyanidin-coated protozoan.

### DETAILED DESCRIPTION

Aspects of the present specification disclose a component. A component disclosed herein may be either synthesized chemically from precursor organic compounds, extracted from a natural source such as a plant, or obtained by a combination of synthesis and extraction. The components disclosed herein have the ability to coat the exposed surfaces of one or more target particles. The coating of a target particle with a component disclosed herein can, without limitation, increase or decrease the immunogenicity of a target particle in a biological organism to which the component-coated target particle is administered, protect a biological organism from the toxic effects of the target particle, exhibit a toxic effect on a biological organism, provide a lubricant effect to the target particle, protect a target particle from degradation such as wear, abrasion, oxidation, or other processes, increase or decrease the electrical conductivity or resistance of a target particle, produce images or other patterns, including graphics and text, on a surface of target particle, increase or decrease the disconnection or desorption of species from a surface of a target particle, increase or decrease the biological activity or action of living organisms, or expose or mask a surface of a target particle from incident radiation or chemical or mechanical treatment.

A plant disclosed herein may be a dicot or a monocot. A plant may be a tree, a vegetable or a vine. In addition, a plant may be a fruit bearing plant, including, without limitation, a plant capable of producing grapes or persimmons. In fact, any fruit capable of providing a component disclosed herein may be used as a source. In an embodiment, a fruit comprises, without limitation, a persimmon or a grape. In an embodiment, preparation of a component from a commercially-available grape-seed (Vitaceae) extract is described in U.S. Patent Publication No. 20100221281, except that the coating so produced contains proanthocyanidins disclosed herein. It will be apparent to one skilled in the art that a wide range of naturally-occurring and synthetic raw materials, which can be sourced from a wide range of plants and animals or feedstock reagents can be used. In one embodiment, commercial grape-seed extract can serve as the source of a component disclosed herein. Additional potential sources include most highly-colored fruits and vegetables, as well as other sources familiar to those skilled in the art.

A component disclosed herein may be a tannin. A tannin disclosed herein may be a pseudotannin or a proanthocyanidin. A tannin (also known as vegetable tannin, natural organic tannins or sometimes tannoid, i.e. a type of biomolecule, as opposed to modern synthetic tannin) is an astringent, bitter plant polyphenolic compound that binds to and precipitates proteins and various other organic compounds including amino acids and alkaloids. A tannin disclosed herein may be naturally occurring or synthetically manufactured. A tannin may be obtained and extracted from a gymnosperm and/or an angiosperm. In aspects of this embodiment, a tannin may be obtained or extracted from Aceraceae, Actinidiaceae, Anacardiaceae, Bixaceae, Burseraceae, Combretaceae, Dipterocarpaceae, Ericaceae, Grossulariaceae, Myricaceae, Najadaceae, Vitaceae and/or Typhaceae. A pseudotannin may be extracted from, without limitation, tea or coffee.

In an embodiment, a tannin disclosed herein have molecular masses or weights ranging from 500 to over 3,000 (including, without limitation, gallic acid esters) and up to 20,000 (including, without limitation, proanthocyanidins) Daltons. In aspects of this embodiment, a tannin has a molecular weight of, e.g., at least 10 Daltons, at least 25 Daltons, at least 50 Daltons, at least 75 Daltons, at least 100 Daltons, at least 200 Daltons, at least 300 Daltons, at least 400 Daltons, at least 500 Daltons, at least 600 Daltons, at least 700 Daltons, at least 800 Daltons, at least 900 Daltons, at least 1000 Daltons, at least 1250 Daltons, at least 1500 Daltons, at least 1750 Daltons, at least 2000 Daltons, at least 2250 Daltons, at least 2500 Daltons, at least 2750 Daltons, at least 3000 Daltons, at least 3250 Daltons, at least 3500 Daltons, at least 3750 Daltons, at least 4000 Daltons, at least 4250 Daltons, at least 4500 Daltons, at least 4750 Daltons, at least 5000 Daltons, at least 5250 Daltons, at least 5500 Daltons, at least 5750 Daltons, 6000 Daltons, 6250 Daltons, 6500 Daltons, at least 6750 Daltons, at least 7000 Daltons, at least 7250 Daltons, at least 7500 Daltons, at least 7750 Daltons, at least 8000 Daltons, at least 8250 Daltons, at least 8500 Daltons, at least 8750 Daltons, at least 9000 Daltons, at least 9250 Daltons, at least 9500 Daltons, at least 9750 Daltons, at least 10000 Daltons, at least 10250 Daltons, at least 10500 Daltons, at least 10750 Daltons, at least 11000 Daltons, at least 11250 Daltons, at least 11500 Daltons, at least 11750 Daltons, at least 12000 Daltons, at least 12250 Daltons, at least 12500 Daltons, at least 12750 Daltons, at least 13000 Daltons, at least 13250 Daltons, at least 13500 Daltons, at least 13750 Daltons, at least 14000 Daltons, at least 14250 Daltons, at least 14500 Daltons, at least 14750 Daltons, at least 15000 Daltons, at least 15250 Daltons, at least 15500 Daltons, at least 15750 Daltons, at least 16000 Daltons, at least 16250 Daltons, at least 16500 Daltons, at least 16750 Daltons, at least 17000 Daltons, at least 17250 Daltons, at least 17500 Daltons, at least 17750 Daltons, at least 18000 Daltons, at least 18250 Daltons, at least 18500 Daltons, at least 18750 Daltons, at least 19000 Daltons, at least 19250 Daltons, at least 19500 Daltons, at least 19750 Daltons, at least 20000 Daltons, at least 20250 Daltons, at least 20500 Daltons, at least 20750 Daltons, at least 21000 Daltons, at least 22000 Daltons, at least 23000 Daltons, at least 24000 Daltons, at least 25000, or more.

In other aspects of this embodiment, a tannin disclosed herein has a molecular weight of, e.g., at most 100 Daltons, at most 200 Daltons, at most 300 Daltons, at most 400 Daltons, at most 500 Daltons, at most 600 Daltons, at most 700 Daltons, at most 800 Daltons, at most 900 Daltons, at most 1000 Daltons, at most 1250 Daltons, at most 1500 Daltons, at most 1750 Daltons, at most 2000 Daltons, at most 2250 Daltons, at most 2500 Daltons, at most 2750 Daltons, at most 3000 Daltons, at most 3250 Daltons, at most 3500 Daltons, at most 3750 Daltons, at most 4000 Daltons, at most 4250 Daltons, at most 4500 Daltons, at most 4750 Daltons, at most 5000 Daltons, at most 5250 Daltons, at most 5500 Daltons, at most 5750 Daltons, 6000 Daltons, 6250 Daltons, 6500 Daltons, at most 6750 Daltons, at most 7000 Daltons, at most 7250 Daltons, at most 7500 Daltons, at most 7750 Daltons, at most 8000 Daltons, at most 8250 Daltons, at most 8500 Daltons, at most 8750 Daltons, at most 9000 Daltons, at most 9250 Daltons, at most 9500 Daltons, at most 9750 Daltons, at most 10000 Daltons, at most 10250 Daltons, at most 10500 Daltons, at most 10750 Daltons, at most 11000 Daltons, at most 11250 Daltons, at most 11500 Daltons, at most 11750 Daltons, at most 12000 Daltons, at most 12250 Daltons, at most 12500 Daltons, at most 12750 Daltons, at most 13000 Daltons, at most 13250 Daltons, at most 13500 Daltons, at most 13750 Daltons, at most 14000 Daltons, at most 14250 Daltons, at most 14500 Daltons, at most 14750 Daltons, at most 15000 Daltons, at most 15250 Daltons, at most 15500 Daltons, at most 15750 Daltons, at most 16000 Daltons, at most 16250 Daltons, at most 16500 Daltons, at most 16750 Daltons, at most 17000 Daltons, at most 17250 Daltons, at most 17500 Daltons, at most 17750 Daltons, at most 18000 Daltons, at most 18250 Daltons, at most 18500 Daltons, at most 18750 Daltons, at most 19000 Daltons, at most 19250 Daltons, at most 19500 Daltons, at most 19750 Daltons, at most 20000 Daltons, at most 20250 Daltons, at most 20500 Daltons, at most 20750 Daltons, at most 21000 Daltons, at most 22000 Daltons, at most 23000 Daltons, at most 24000 Daltons, at most 25000 Daltons.

In other aspects of this embodiment, a tannin disclosed herein has a molecular weight of, e.g., about 100 Daltons to about 500 Daltons, about 100 Daltons to about 1000 Daltons, about 100 Daltons to about 1500 Daltons, about 100 Daltons to about 2000 Daltons, about 100 Daltons to about 2500 Daltons, about 100 Daltons to about 3000 Daltons, about 100 Daltons to about 3500 Daltons, about 100 Daltons to about 4000 Daltons, about 100 Daltons to about 4500 Daltons, about 100 Daltons to about 5000 Daltons, about 100 Daltons to about 5500 Daltons, about 100 Daltons to about 6000 Daltons, about 100 Daltons to about 6500 Daltons, about 100 Daltons to about 7000 Daltons, about 100 Daltons to about 7500 Daltons, about 100 Daltons to about 8000 Daltons, about 100 Daltons to about 8500 Daltons, about 100 Daltons to about 9000 Daltons, about 100 Daltons to about 9500 Daltons, about 100 Daltons to about 10000 Daltons, about 100 Daltons to about 10500 Daltons, about 100 Daltons to about 11000 Daltons, about 100 Daltons to about 11500 Daltons, about 100 Daltons to about 12000 Daltons, about 100 Daltons to about 12500 Daltons, about 100 Daltons to about 13000 Daltons, about 100 Daltons to about 13500 Daltons, about 100 Daltons to about 14000 Daltons, about 100 Daltons to about 14500 Daltons, about 100 Daltons to about 15000 Daltons, about 100 Daltons to about 15500 Daltons, about 100 Daltons to about 16000 Daltons, about 100 Daltons to about 16500 Daltons, about 100 Daltons to about 17000 Daltons, about 100 Daltons to about 17500 Daltons, about 100 Daltons to about 18000 Daltons, about 100 Daltons to about 18500 Daltons, about 100 Daltons to about 19000 Daltons, about 100 Daltons to about 19500 Daltons, about 100 Daltons to about 20000 Daltons, about 100 Daltons to about 20500 Daltons, about 100 Daltons to about 21000 Daltons, about 100 Daltons to about 21500 Daltons, about 100 Daltons to about 22000 Daltons, about 100 Daltons to about 22500 Daltons, about 100 Daltons to about 23000 Daltons, about 100 Daltons to about 23500 Daltons, about 100 Daltons to about 24000 Daltons, about 100 Daltons to about 24500 Daltons, about 100 Daltons to about 25000 Daltons.

In other aspects of this embodiment, a tannin disclosed herein has a molecular weight of, e.g., about 1000 Daltons to about 1500 Daltons, about 1000 Daltons to about 2000 Daltons, about 1000 Daltons to about 2500 Daltons, about 1000 Daltons to about 3000 Daltons, about 1000 Daltons to about 3500 Daltons, about 1000 Daltons to about 4000 Daltons, about 1000 Daltons to about 4500 Daltons, about 1000 Daltons to about 5000 Daltons, about 1000 Daltons to about 5500 Daltons, about 1000 Daltons to about 6000 Daltons, about 1000 Daltons to about 6500 Daltons, about 1000 Daltons to about 7000 Daltons, about 1000 Daltons to about 7500 Daltons, about 1000 Daltons to about 8000 Daltons, about 1000 Daltons to about 8500 Daltons, about 1000 Daltons to about 9000 Daltons, about 1000 Daltons to about 9500 Daltons, about 1000 Daltons to about 10000 Daltons, about 1000 Daltons to about 10500 Daltons, about 1000 Daltons to about 11000 Daltons, about 1000 Daltons to about 11500 Daltons, about 1000 Daltons to about 12000 Daltons, about 1000 Daltons to about 12500 Daltons, about 1000 Daltons to about 13000 Daltons, about 1000 Daltons to about 13500 Daltons, about 1000 Daltons to about 14000 Daltons, about 1000 Daltons to about 14500 Daltons, about 1000 Daltons to about 15000 Daltons, about 1000 Daltons to about 15500 Daltons, about 1000 Daltons to about 16000 Daltons, about 1000 Daltons to about 16500 Daltons, about 1000 Daltons to about 17000 Daltons, about 1000 Daltons to about 17500 Daltons, about 1000 Daltons to about 18000 Daltons, about 1000 Daltons to about 18500 Daltons, about 1000 Daltons to about 19000 Daltons, about 1000 Daltons to about 19500 Daltons, about 1000 Daltons to about 20000 Daltons, about 1000 Daltons to about 20500 Daltons, about 1000 Daltons to about 21000 Daltons, about 1000 Daltons to about 21500 Daltons, about 1000 Daltons to about 22000 Daltons, about 1000 Daltons to about 22500 Daltons, about 1000 Daltons to about 23000 Daltons, about 1000 Daltons to about 23500 Daltons, about 1000 Daltons to about 24000 Daltons, about 1000 Daltons to about 24500 Daltons, about 1000 Daltons to about 25000 Daltons.

In other aspects of this embodiment, a tannin disclosed herein has a molecular weight of, e.g., about 2500 Daltons to about 3000 Daltons, about 2500 Daltons to about 3500 Daltons, about 2500 Daltons to about 4000 Daltons, about 2500 Daltons to about 4500 Daltons, about 2500 Daltons to about 5000 Daltons, about 2500 Daltons to about 5500 Daltons, about 2500 Daltons to about 6000 Daltons, about 2500 Daltons to about 6500 Daltons, about 2500 Daltons to about 7000 Daltons, about 2500 Daltons to about 7500 Daltons, about 2500 Daltons to about 8000 Daltons, about 2500 Daltons to about 8500 Daltons, about 2500 Daltons to about 9000 Daltons, about 2500 Daltons to about 9500 Daltons, about 2500 Daltons to about 10000 Daltons, about 2500 Daltons to about 10500 Daltons, about 2500 Daltons to about 11000 Daltons, about 2500 Daltons to about 11500 Daltons, about 2500 Daltons to about 12000 Daltons, about 2500 Daltons to about 12500 Daltons, about 2500 Daltons to about 13000 Daltons, about 2500 Daltons to about 13500 Daltons, about 2500 Daltons to about 14000 Daltons, about 2500 Daltons to about 14500 Daltons, about 2500 Daltons to about 15000 Daltons, about 2500 Daltons to about 15500 Daltons, about 2500 Daltons to about 16000 Daltons, about 2500 Daltons to about 16500 Daltons, about 2500 Daltons to about 17000 Daltons, about 2500 Daltons to about 17500 Daltons, about 2500 Daltons to about 18000 Daltons, about 2500 Daltons to about 18500 Daltons, about 2500 Daltons to about 19000 Daltons, about 2500 Daltons to about 19500 Daltons, about 2500 Daltons to about 20000 Daltons, about 2500 Daltons to about 20500 Daltons, about 2500 Daltons to about 21000 Daltons, about 2500 Daltons to about 21500 Daltons, about 2500 Daltons to about 22000 Daltons, about 2500 Daltons to about 22500 Daltons, about 2500 Daltons to about 23000 Daltons, about 2500 Daltons to about 23500 Daltons, about 2500 Daltons to about 24000 Daltons, about 2500 Daltons to about 24500 Daltons, about 2500 Daltons to about 25000 Daltons.

In other aspects of this embodiment, a tannin disclosed herein has a molecular weight of, e.g., about 5000 Daltons to about 5500 Daltons, about 5000 Daltons to about 6000 Daltons, about 5000 Daltons to about 6500 Daltons, about 5000 Daltons to about 7000 Daltons, about 5000 Daltons to about 7500 Daltons, about 5000 Daltons to about 8000 Daltons, about 5000 Daltons to about 8500 Daltons, about 5000 Daltons to about 9000 Daltons, about 5000 Daltons to about 9500 Daltons, about 5000 Daltons to about 10000 Daltons, about 5000 Daltons to about 10500 Daltons, about 5000 Daltons to about 11000 Daltons, about 5000 Daltons to about 11500 Daltons, about 100 Daltons to about 12000 Daltons, about 5000 Daltons to about 12500 Daltons, about 5000 Daltons to about 13000 Daltons, about 5000 Daltons to about 13500 Daltons, about 5000 Daltons to about 14000 Daltons, about 5000 Daltons to about 14500 Daltons, about 5000 Daltons to about 15000 Daltons, about 5000 Daltons to about 15500 Daltons, about 5000 Daltons to about 16000 Daltons, about 5000 Daltons to about 16500 Daltons, about 5000 Daltons to about 17000 Daltons, about 5000 Daltons to about 17500 Daltons, about 5000 Daltons to about 18000 Daltons, about 5000 Daltons to about 18500 Daltons, about 5000 Daltons to about 19000 Daltons, about 5000 Daltons to about 19500 Daltons, about 5000 Daltons to about 20000 Daltons, about 5000 Daltons to about 20500 Daltons, about 5000 Daltons to about 21000 Daltons, about 5000 Daltons to about 21500 Daltons, about 5000 Daltons to about 22000 Daltons, about 5000 Daltons to about 22500 Daltons, about 5000 Daltons to about 23000 Daltons, about 5000 Daltons to about 23500 Daltons, about 5000 Daltons to about 24000 Daltons, about 5000 Daltons to about 24500 Daltons, about 5000 Daltons to about 25000 Daltons.

In other aspects of this embodiment, a tannin disclosed herein has a molecular weight of, e.g., about 10000 Daltons to about 10500 Daltons, about 10000 Daltons to about 11000 Daltons, about 10000 Daltons to about 11500 Daltons, about 10000 Daltons to about 12000 Daltons, about 10000 Daltons to about 12500 Daltons, about 10000 Daltons to about 13000 Daltons, about 10000 Daltons to about 13500 Daltons, about 10000 Daltons to about 14000 Daltons, about 10000 Daltons to about 14500 Daltons, about 10000 Daltons to about 15000 Daltons, about 10000 Daltons to about 15500 Daltons, about 10000 Daltons to about 16000 Daltons, about 10000 Daltons to about 16500 Daltons, about 10000 Daltons to about 17000 Daltons, about 10000 Daltons to about 17500 Daltons, about 10000 Daltons to about 18000 Daltons, about 10000 Daltons to about 18500 Daltons, about 10000 Daltons to about 19000 Daltons, about 10000 Daltons to about 19500 Daltons, about 10000 Daltons to about 20000 Daltons, about 10000 Daltons to about 20500 Daltons, about 10000 Daltons to about 21000 Daltons, about 10000 Daltons to about 21500 Daltons, about 10000 Daltons to about 22000 Daltons, about 10000 Daltons to about 22500 Daltons, about 10000 Daltons to about 23000 Daltons, about 10000 Daltons to about 23500 Daltons, about 10000 Daltons to about 24000 Daltons, about 10000 Daltons to about 24500 Daltons, about 10000 Daltons to about 25000 Daltons.

In other aspects of this embodiment, a tannin disclosed herein has a molecular weight of, e.g., about 15000 Daltons to about 15500 Daltons, about 15000 Daltons to about 16000 Daltons, about 15000 Daltons to about 16500 Daltons, about 15000 Daltons to about 17000 Daltons, about 15000 Daltons to about 17500 Daltons, about 15000 Daltons to about 18000 Daltons, about 15000 Daltons to about 18500 Daltons, about 15000 Daltons to about 19000 Daltons, about 15000 Daltons to about 19500 Daltons, about 15000 Daltons to about 20000 Daltons, about 15000 Daltons to about 20500 Daltons, about 15000 Daltons to about 21000 Daltons, about 15000 Daltons to about 21500 Daltons, about 15000 Daltons to about 22000 Daltons, about 15000 Daltons to about 22500 Daltons, about 15000 Daltons to about 23000 Daltons, about 15000 Daltons to about 23500 Daltons, about 15000 Daltons to about 24000 Daltons, about 15000 Daltons to about 24500 Daltons, about 15000 Daltons to about 25000 Daltons.

In an embodiment, a tannin may be a proanthocyanidin. Proanthocyanidines are a class of polyphenols found in a variety of plants. A proanthocyanidin is an oligomeric flavonoid having a polymer length of 2 to 100 (or more) flavan-3-ol units joined by carbon-carbon or carbon-oxygen bonds that are not readily susceptible to being cleaved by hydrolysis. In aspects of this embodiment, a proanthocyanidin may be a polymer of, e.g., 2 or more, 3 or more, 4 or more, 5 or more, 6 or more, 7 or more, 8 or more, 9 or more, 10 or more, 15 or more, 20 or more, 25 or more, 30 or more, 35 or more, 40 or more, 45 or more, 50 or more, 55 or more, 60 or more, 65 or more, 70 or more, 85 or more, 90 or more, 95 or more, flavan-3-ol units joined by carbon-carbon or carbon-oxygen bonds that are not readily susceptible to being cleaved by hydrolysis. In other aspects of this embodiment, a proanthocyanidin may be a polymer of, e.g., at least 2, at least 5, at least 10, at least 15, at least 20, at least 35, at least 30, at least 35, at least 40, at least 45, at least 50, at least 55, at least 60, at least 65, at least 70, at least 75, at least 80, at least 85, at least 90 at least 95, at least 97, flavan-3-ol units joined by carbon-carbon or carbon-oxygen bonds that are not readily susceptible to being cleaved by hydrolysis. In other aspects of this embodiment, a proanthocyanidin may be a polymer of, e.g., at most 2, at most 5, at most 10, at most 15, at most 20, at most 35, at most 30, at most 35, at most 40, at most 45, at most 50, at most 55, at most 60, at most 65, at most 70, at most 75, at most 80, at most 85, at most 90 at most 95, at most 97, flavan-3-ol units joined by carbon-carbon or carbon-oxygen bonds that are not readily susceptible to being cleaved by hydrolysis.

In other aspects of this embodiment, a proanthocyanidin may be of a polymer of, e.g., about 2 to about 10, about 2 to about 15, about 2 to about 20, about 2 to about 25, about 2 to about 30, about 2 to about 35, about 2 to about 40, about 2 to about 45, about 2 to about 50, about 2 to about 55, about 2 to about 60, about 2 to about 65, about 2 to about 70, about 2 to about 75, about 2 to about 80, about 2 to about 85, about 2 to about 90, about 2 to about 95, or about 2 to about 100 flavan-3-ol units joined by bonds which are not susceptible to being cleaved by hydrolysis.

In other aspects of this embodiment, a proanthocyanidin may be of a polymer of, e.g., about 5 to about 10, about 5 to about 15, about 5 to about 20, about 5 to about 25, about 5 to about 30, about 5 to about 35, about 5 to about 40, about 5 to about 45, about 5 to about 50, about 5 to about 55, about 5 to about 60, about 5 to about 65, about 5 to about 70, about 5 to about 75, about 5 to about 80, about 5 to about 85, about 5 to about 90, about 5 to about 95, or about 5 to about 100 flavan-3-ol units joined by bonds which are not susceptible to being cleaved by hydrolysis.

In other aspects of this embodiment, a proanthocyanidin may be of a polymer of, e.g., about 10 to about 15, about 10 to about 20, about 10 to about 25, about 10 to about 30, about 10 to about 35, about 10 to about 40, about 10 to about 45, about 10 to about 50, about 10 to about 55, about 10 to about 60, about 10 to about 65, about 10 to about 70, about 10 to about 75, about 10 to about 80, about 10 to about 85, about 10 to about 90, about 10 to about 95, or about 10 to about 100 flavan-3-ol units joined by bonds which are not susceptible to being cleaved by hydrolysis.

In other aspects of this embodiment, a proanthocyanidin may be of a polymer of, e.g., about 15 to about 20, about 15 to about 25, about 15 to about 30, about 15 to about 35, about 15 to about 40, about 15 to about 45, about 15 to about 50, about 15 to about 55, about 15 to about 60, about 15 to about 65, about 15 to about 70, about 15 to about 75, about 15 to about 80, about 15 to about 85, about 15 to about 90, about 15 to about 95, or about 15 to about 100 flavan-3-ol units joined by bonds which are not susceptible to being cleaved by hydrolysis.

In other aspects of this embodiment, a proanthocyanidin may be of a polymer of, e.g., about 20 to about 25, about 20 to about 30, about 20 to about 35, about 20 to about 40, about 20 to about 45, about 20 to about 50, about 20 to about 55, about 20 to about 60, about 20 to about 65, about 20 to about 70, about 20 to about 75, about 20 to about 80, about 20 to about 85, about 20 to about 90, about 20 to about 95, or about 20 to about 100 flavan-3-ol units joined by bonds which are not susceptible to being cleaved by hydrolysis.

In other aspects of this embodiment, a proanthocyanidin may be of a polymer of, e.g., about 25 to about 30, about 25 to about 35, about 25 to about 40, about 25 to about 45, about 25 to about 50, about 25 to about 55, about 25 to about 60, about 25 to about 65, about 25 to about 70, about 25 to about 75, about 25 to about 80, about 25 to about 85, about 25 to about 90, about 25 to about 95, or about 25 to about 100 flavan-3-ol units joined by bonds which are not susceptible to being cleaved by hydrolysis.

In other aspects of this embodiment, a proanthocyanidin may be of a polymer of, e.g., about 30 to about 35, about 30 to about 40, about 30 to about 45, about 30 to about 50, about 30 to about 55, about 30 to about 60, about 30 to about 65, about 30 to about 70, about 30 to about 75, about 30 to about 80, about 30 to about 85, about 30 to about 90, about 30 to about 95, or about 30 to about 100 flavan-3-ol units joined by bonds which are not susceptible to being cleaved by hydrolysis.

In other aspects of this embodiment, a proanthocyanidin may be of a polymer of, e.g., about 40 to about 45, about 40 to about 50, about 40 to about 55, about 40 to about 60, about 40 to about 65, about 40 to about 70, about 40 to about 75, about 40 to about 80, about 40 to about 85, about 40 to about 90, about 40 to about 95, or about 40 to about 100 flavan-3-ol units joined by bonds which are not susceptible to being cleaved by hydrolysis.

In other aspects of this embodiment, a proanthocyanidin may be of a polymer of, e.g., about 50 to about 55, about 50 to about 60, about 50 to about 65, about 50 to about 70, about 50 to about 75, about 50 to about 80, about 50 to about 85, about 50 to about 90, about 50 to about 95, or about 50 to about 100 flavan-3-ol units joined by bonds which are not susceptible to being cleaved by hydrolysis.

In other aspects of this embodiment, a proanthocyanidin may be of a polymer of, e.g., about 60 to about 65, about 60 to about 70, about 60 to about 75, about 60 to about 80, about 60 to about 85, about 60 to about 90, about 60 to about 95, or about 60 to about 100 flavan-3-ol units joined by bonds which are not susceptible to being cleaved by hydrolysis.

In other aspects of this embodiment, a proanthocyanidin may be of a polymer of, e.g., about 70 to about 75, about 70 to about 80, about 70 to about 85, about 70 to about 90, about 70 to about 95, or about 70 to about 100 flavan-3-ol units joined by bonds which are not susceptible to being cleaved by hydrolysis.

In other aspects of this embodiment, a proanthocyanidin may be of a polymer of, e.g., about 80 to about 85, about 80 to about 90, about 80 to about 95, or about 80 to about 100 flavan-3-ol units joined by bonds which are not susceptible to being cleaved by hydrolysis.

In other aspects of this embodiment, a proanthocyanidin may be of a polymer of, e.g., about 90 to about 95, about 90 to about 100, or about 95 to about 100 flavan-3-ol units joined by bonds which are not susceptible to being cleaved by hydrolysis.

A component disclosed herein, including without limitation, a tannin, a pseudotannin, and/or a proanthocyanidin has the capability to adhere to a scaffold. A scaffold is any structure to which a component disclosed herein can adhere. A scaffold includes, without limitation, a particulate lattice. A scaffold can be obtained from any source, whether that is a commercial source such as the many firms which can supply particles and materials in a broad array of sizes, shapes, and compositions, or else a scaffold produced in the same facility explicitly for the purpose of coating it, or even a scaffold which is co-produced along with the coating process. In an embodiment, a scaffold constructed of the same material used to coat such scaffold is, without limitation, a particulate lattice. If the particulate lattice is produced separately, then the coating material can be obtained, without limitation, from any convenient source including, without limitation, the extraction of a tannin, a pseudotannin, and/or a proanthocyanidin from plant sources using sub-critical water or sub- or super-critical fluid extraction as any of the many such methods apparent to one skilled in the art, including, without limitation, those disclosed in, for example, R. Murga et al., J. Agric. Food Chem. 48, 3408 (2000), R. Prior et al., J. Agric. Food Chem. 49, 1270 (2001), L. Gu et al., J. Agric. Food Chem. 50, 4852 (2002), and N. Kohler, V. Wray, P. Winterhalter, J. Chromatogr. A 1177, 114 (2008), and T-I Lafka, V. Sinanoglu, and E. Lazos, Food Chem 104, 1206 (2007).

Aspects of the present specification disclose in part a scaffold. A scaffold disclosed herein may be either synthesized chemically from precursor organic compounds, extracted from a natural source such as a plant, or obtained by a combination of synthesis and extraction. A scaffold disclosed herein have the ability to facilitate the coating of exposed surfaces of one or more target particles by one or more components disclosed herein.

A plant disclosed herein may be a dicot or a monocot. A plant may be a tree, a vegetable or a vine. In addition, a plant may be a fruit bearing plant, including, without limitation, a plant capable of producing grapes or persimmons. In fact, any fruit capable of providing a scaffold disclosed herein may be used as a source. In an embodiment, a fruit comprises, without limitation, a persimmon or a grape. It will be apparent to one skilled in the art that a wide range of naturally-occurring and synthetic raw materials, which can be sourced from a wide range of plants and animals or feedstock reagents can be used. In one embodiment, commercial grape-seed extract can serve as the source of a scaffold disclosed herein. Additional potential sources include most highly-colored fruits and vegetables, as well as other sources familiar to those skilled in the art.

A scaffold disclosed herein may be a self-assembled scaffold of a component-coated scaffold. A component disclosed herein, including a tannin, a psuedotannin or a proanthocyanidin may be present not just as individual molecules but rather aggregate or form a larger structure referred to as a self-assembled scaffold. Additionally or alternatively, a component disclosed herein, including a tannin, a psuedotannin or a proanthocyanidin may associate with a separate scaffold material used as a support lattice or particulate lattice for the components disclosed herein, thereby forming a component-coated scaffold. Thus, a scaffold comprises a particulate lattice includes a component disclosed herein that is, without limitation, identical or similar to, or different from, the composition of the scaffold itself.

In an embodiment, a scaffold includes, without limitation, a support lattice or particulate lattice comprising one or more components disclosed herein. In as aspect of this embodiment, a scaffold includes, without limitation, a support lattice or particulate lattice comprising a tannin, a psuedotannin and/or a proanthocyanidin

In another embodiment, a scaffold includes, without limitation, a support lattice or particulate lattice comprising a biological organism, a viral particle, a viron, a protein, a protein cage, a peptide, an antibody, a venom, a toxin, a vault, a nucleic acid, a fat, a lipid, a micelle, a liposome, a carbohydrate, a therapeutic compound, an active ingredient, a polymer latex, a metal object, a composite object, a fiber, a bead, and/or a rubber object. In an embodiment, a scaffold is, without limitation, a bead. In an aspect of this embodiment, a bead is, without limitation, a glass bead, a Sephadex bead, a dextran bead, a poly(styrene) bead, a silica bead, and/or a diethylaminoethylcellulose bead. In another aspect of this embodiment, a scaffold is a carbohydrate support lattice or a carbohydrate particulate lattice.

In an embodiment, a scaffold disclosed herein may be any geometrical three-dimensional shape. In aspects of this embodiment, a scaffold may be spherical, ovoidal, circular, cubical, conal, rectangular, cylindrical, helical, triangular, pyramidal, and/or tetrahedral, any other type of polyhedron, or other three-dimensional shape. In aspects of this embodiment, a scaffold may be a cubical prism, a rectangular prism, a triangular prism, a hexagonal prism, an icosahedron, a square pyramid, a rectangular pyramid, a triangular pyramid, a hexagonal pyramid, a round cylinder, or a square cylinder.

A surface of the scaffold disclosed herein provides, without limitation, a high degree of curvature, which is capable of accelerating the transfer of a component disclosed herein to another surface, such as, e.g., the surface of a target particle. In addition, the size of a scaffold disclosed herein can, without limitation, increase the efficiency of transfer of a component disclosed herein to a surface of a target particle. In an embodiment, a scaffold may be any nanoscale to microscale size. In aspects of this embodiment, a scaffold including, without limitation, a particulate lattice, has a diameter of, e.g., at least 1 nm (nanometer), at least 2 nm, at least 3 nm, at least 4 nm, at least 5 nm, at least 6 nm, at least 7 nm, at least 8 nm, at least 9 nm, at least 10 nm, at least 11 nm, at least 12 nm, at least 13 nm, at least 14 nm, at least 15 nm, at least 16 nm, at least 17 nm, at least 18 nm, at least 19 nm, at least 20 nm, at least 21 nm, at least 22 nm, at least 23 nm, at least 24 nm, at least 25 nm, at least 26 nm, at least 27 nm, at least 28 nm, at least 29 nm, at least 30 nm, at least 31 nm, at least 32 nm, at least 33 nm, at least 34 nm, at least 35 nm, at least 36 nm, at least 37 nm, at least 38 nm, at least 39 nm, at least 40 nm, at least 41 nm, at least 42 nm, at least 43 nm, at least 44 nm, at least 45 nm, at least 46 nm, at least 47 nm, at least 48 nm, at least 49 nm, at least 50 nm, at least 51 nm, at least 52 nm, at least 53 nm, at least 54 nm, at least 55 nm, at least 56 nm, at least 57 nm, at least 58 nm, at least 59 nm, at least 60 nm, at least 61 nm, at least 62 nm, at least 63 nm, at least 64 nm, at least 65 nm, at least 66 nm, at least 67 nm, at least 68 nm, at least 69 nm, at least 70 nm, at least 71 nm, at least 72 nm, at least 73 nm, at least 74 nm, at least 75 nm, at least 76 nm, at least 77 nm, at least 78 nm, at least 79 nm, at least 80 nm, at least 81 nm, at least 82 nm, at least 83 nm, at least 84 nm, at least 85 nm, at least 86 nm, at least 87 nm, at least 88 nm, at least 89 nm, at least 90 nm, at least 91 nm, at least 92 nm, at least 93 nm, at least 94 nm, at least 95 nm, at least 96 nm, at least 97 nm, at least 98 nm, at least 99 nm, at least 100 nm, at least 150 nm, at least 200 nm, at least 250 nm, at least 300 nm, at least 350 nm, at least 400 nm, at least 450 nm, at least, 0.5 μm (micrometers), at least 1.0 μm, at least 2 μm, at least 3 μm, at least 4 μm, at least 5 μm, at least 6 μm, at least 7 μm, at least 8 μm, at least 9 μm, at least 10 μm, at least 11 μm, at least 12 μm, at least 13 μm, at least 14 μm, at least 15 μm, at least 16 μm, at least 17 μm, at least 18 μm, at least 19 μm, at least 20 μm, at least 21 μm, at least 22 μm, at least 23 μm, at least 24 μm, at least 25 μm, at least 26 μm, at least 27 μm, at least 28 μm, at least 29 μm, at least 30 μm, at least 31 μm, at least 32 μm, at least 33 μm, at least 34 μm, at least 35 μm, at least 36 μm, at least 37 μm, at least 38 μm, at least 39 μm, at least 40 μm, at least 41 μm, at least 42 μm, at least 43 μm, at least 44 μm, at least 45 μm, at least 46 μm, at least 47 μm, at least 48 μm, at least 49 μm, at least 50 μm, at least 51 μm, at least 52 μm, at least 53 μm, at least 54 μm, at least 55 μm, at least 56 μm, at least 57 μm, at least 58 μm, at least 59 μm, at least 60 μm, at least 61 μm, at least 62 μm, at least 63 μm, at least 64 μm, at least 65 μm, at least 66 μm, at least 67 μm, at least 68 μm, at least 69 μm, at least 70 μm, at least 71 μm, at least 72 μm, at least 73 μm, at least 74 μm, at least 75 μm, at least 76 μm, at least 77 μm, at least 78 μm, at least 79 μm, at least 80 μm, at least 81 μm, at least 82 μm, at least 83 μm, at least 84 μm, at least 85 μm, at least 86 μm, at least 87 μm, at least 88 μm, at least 89 μm, at least 90 μm, at least 91 μm, at least 92 μm, at least 93 μm, at least 94 μm, at least 95 μm, at least 96 μm, at least 97 μm, at least 98 μm, at least 99 μm, at least 100 μm, or more.

In other aspects of this embodiment, a scaffold including, without limitation, a particulate lattice, has a diameter of, e.g., at most 1 nm (nanometer), at most 2 nm, at most 3 nm, at most 4 nm, at most 5 nm, at most 6 nm, at most 7 nm, at most 8 nm, at most 9 nm, at most 10 nm, at most 11 nm, at most 12 nm, at most 13 nm, at most 14 nm, at most 15 nm, at most 16 nm, at most 17 nm, at most 18 nm, at most 19 nm, at most 20 nm, at most 21 nm, at most 22 nm, at most 23 nm, at most 24 nm, at most 25 nm, at most 26 nm, at most 27 nm, at most 28 nm, at most 29 nm, at most 30 nm, at most 31 nm, at most 32 nm, at most 33 nm, at most 34 nm, at most 35 nm, at most 36 nm, at most 37 nm, at most 38 nm, at most 39 nm, at most 40 nm, at most 41 nm, at most 42 nm, at most 43 nm, at most 44 nm, at most 45 nm, at most 46 nm, at most 47 nm, at most 48 nm, at most 49 nm, at most 50 nm, at most 51 nm, at most 52 nm, at most 53 nm, at most 54 nm, at most 55 nm, at most 56 nm, at most 57 nm, at most 58 nm, at most 59 nm, at most 60 nm, at most 61 nm, at most 62 nm, at most 63 nm, at most 64 nm, at most 65 nm, at most 66 nm, at most 67 nm, at most 68 nm, at most 69 nm, at most 70 nm, at most 71 nm, at most 72 nm, at most 73 nm, at most 74 nm, at most 75 nm, at most 76 nm, at most 77 nm, at most 78 nm, at most 79 nm, at most 80 nm, at most 81 nm, at most 82 nm, at most 83 nm, at most 84 nm, at most 85 nm, at most 86 nm, at most 87 nm, at most 88 nm, at most 89 nm, at most 90 nm, at most 91 nm, at most 92 nm, at most 93 nm, at most 94 nm, at most 95 nm, at most 96 nm, at most 97 nm, at most 98 nm, at most 99 nm, at most 100 nm, at most 150 nm, at most 200 nm, at most 250 nm, at most 300 nm, at most 350 nm, at most 400 nm, at most 450 nm, at most, 0.5 μm (micrometers), at most 1.0 μm, at most 2 μm, at most 3 μm, at most 4 μm, at most 5 μm, at most 6 μm, at most 7 μm, at most 8 μm, at most 9 μm, at most 10 μm, at most 11 μm, at most 12 μm, at most 13 μm, at most 14 μm, at most 15 μm, at most 16 μm, at most 17 μm, at most 18 μm, at most 19 μm, at most 20 μm, at most 21 μm, at most 22 μm, at most 23 μm, at most 24 μm, at most 25 μm, at most 26 μm, at most 27 μm, at most 28 μm, at most 29 μm, at most 30 μm, at most 31 μm, at most 32 μm, at most 33 μm, at most 34 μm, at most 35 μm, at most 36 μm, at most 37 μm, at most 38 μm, at most 39 μm, at most 40 μm, at most 41 μm, at most 42 μm, at most 43 μm, at most 44 μm, at most 45 μm, at most 46 μm, at most 47 μm, at most 48 μm, at most 49 μm, at most 50 μm, at most 51 μm, at most 52 μm, at most 53 μm, at most 54 μm, at most 55 μm, at most 56 μm, at most 57 μm, at most 58 μm, at most 59 μm, at most 60 μm, at most 61 μm, at most 62 μm, at most 63 μm, at most 64 μm, at most 65 μm, at most 66 μm, at most 67 μm, at most 68 μm, at most 69 μm, at most 70 μm, at most 71 μm, at most 72 μm, at most 73 μm, at most 74 μm, at most 75 μm, at most 76 μm, at most 77 μm, at most 78 μm, at most 79 μm, at most 80 μm, at most 81 μm, at most 82 μm, at most 83 μm, at most 84 μm, at most 85 μm, at most 86 μm, at most 87 μm, at most 88 μm, at most 89 μm, at most 90 μm, at most 91 μm, at most 92 μm, at most 93 μm, at most 94 μm, at most 95 μm, at most 96 μm, at most 97 μm, at most 98 μm, at most 99 μm, at most 100 μm.

In other aspects of this embodiment, a scaffold including, without limitation, a particulate lattice, has a diameter of, e.g., about 1 nm to about 10 nm, about 1 nm to about 15 nm, about 1 nm to about 20 nm, about 1 nm to about 25 nm, about 1 nm to about 30 nm, about 1 nm to about 35 nm, about 1 nm to about 40 nm, about 1 nm to about 45 nm, about 1 nm to about 50 nm, about 1 nm to about 60 nm, about 1 nm to about 70 nm, about 1 nm to about 80 nm, about 1 nm to about 90 nm, about 1 nm to about 100 nm, about 1 nm to about 200 nm, about 1 nm to about 300 nm, about 1 nm to about 400 nm, about 1 nm to about 500 nm, about 1 nm to about 600 nm, about 1 nm to about 700 nm, about 1 nm to about 800 nm, about 1 nm to about 900 nm, about 1 nm to about 1000 nm, about 5 nm to about 10 nm, about 5 nm to about 15 nm, about 5 nm to about 20 nm, about 5 nm to about 25 nm, about 5 nm to about 30 nm, about 5 nm to about 35 nm, about 5 nm to about 40 nm, about 5 nm to about 45 nm, about 5 nm to about 50 nm, about 5 nm to about 60 nm, about 5 nm to about 70 nm, about 5 nm to about 80 nm, about 5 nm to about 90 nm, about 5 nm to about 100 nm, about 5 nm to about 200 nm, about 5 nm to about 300 nm, about 5 nm to about 400 nm, about 5 nm to about 500 nm, about 5 nm to about 600 nm, about 5 nm to about 700 nm, about 5 nm to about 800 nm, about 5 nm to about 900 nm, about 5 nm to about 1000 nm, about 10 nm to about 20 nm, about 10 nm to about 25 nm, about 10 nm to about 30 nm, about 10 nm to about 35 nm, about 10 nm to about 40 nm, about 10 nm to about 45 nm, about 10 nm to about 50 nm, about 10 nm to about 60 nm, about 10 nm to about 70 nm, about 10 nm to about 80 nm, about 10 nm to about 90 nm, about 10 nm to about 100 nm, about 10 nm to about 200 nm, about 10 nm to about 300 nm, about 10 nm to about 400 nm, about 10 nm to about 500 nm, about 10 nm to about 600 nm, about 10 nm to about 700 nm, about 10 nm to about 800 nm, about 10 nm to about 900 nm, about 10 nm to about 1000 nm, about 50 nm to about 100 nm, about 50 nm to about 200 nm, about 50 nm to about 300 nm, about 50 nm to about 400 nm, about 50 nm to about 500 nm, about 50 nm to about 600 nm, about 50 nm to about 700 nm, about 50 nm to about 800 nm, about 50 nm to about 900 nm, about 50 nm to about 1000 nm, about 100 nm to about 200 nm, about 100 nm to about 300 nm, about 100 nm to about 400 nm, about 100 nm to about 500 nm, about 100 nm to about 600 nm, about 100 nm to about 700 nm, about 100 nm to about 800 nm, about 100 nm to about 900 nm, or about 100 nm to about 1000 nm,

In other aspects of this embodiment, a scaffold including, without limitation, a particulate lattice, has a diameter of, e.g., about 1 μm to about 10 μm, about 1 μm to about 15 μm, about 1 μm to about 20 μm, about 1 μm to about 25 μm, about 1 μm to about 30 μm, about 1 μm to about 35 μm, about 1 μm to about 40 μm, about 1 μm to about 45 μm, about 1 μm to about 50 μm, about 1 μm to about 60 μm, about 1 μm to about 70 μm, about 1 μm to about 80 μm, about 1 nm to about 90 μm, or about 1 μm to about 100 μm, about 10 μm to about 15 μm, about 10 μm to about 20 μm, about 10 μm to about 25 μm, about 10 μm to about 30 μm, about 10 μm to about 35 μm, about 10 μm to about 40 μm, about 10 μm to about 45 μm, about 10 μm to about 50 μm, about 10 μm to about 60 μm, about 10 μm to about 70 μm, about 10 μm to about 80 μm, about 10 nm to about 90 μm, or about 10 μm to about 100 μm.

Aspects of the present specification disclose a method for forming a scaffold and transferring to it one or more components disclosed herein. A method of forming a scaffold may be performed by choosing one or more a suitable materials for a scaffold; choosing one or more suitable components disclosed herein; adding the components to a reaction mixture sequentially or else mixing the scaffold and components together all at once; rapidly dialyzing the mixture to cause the formation of the particulate scaffold; and isolating or using the resultant particulate scaffold.

Aspects of the present specification disclose a method for efficiently coating target particles. A method for efficiently coating target particles disclosed herein may be performed by first mixing a component disclosed herein with a separate scaffold disclosed herein to form a component-coated scaffold. The component-coated scaffold may then be exposed to a surface of a target particle. During this exposure, the component disclosed herein is transferred from the scaffold to the surface of the target particle in a manner which is more efficient than would otherwise be possible by exposing the component disclosed herein alone to the surface of the target particle. In other words, the presence of the scaffold facilitates a more effective transfer of a component disclosed herein to the surface of a target particle. This methodology is general, and thus has wide applicability to a broad range of industries in which it is desired to accurately and efficiently coat target surfaces.

In an embodiment, a method for coating a surface comprises a) producing, dissolving, or suspending a scaffold of an appropriate size and composition in a suitable solvent; b) mixing or loading one or more component disclosed herein onto the dissolved or suspended scaffold to form a component-coated scaffold; c) exposing the component-coated scaffold to a surface of an appropriate-sized target particle; and d) transferring at least some of the component onto a surface of the target particle.

In an embodiment, a method for coating a surface comprises a) producing, dissolving, or suspending a scaffold disclosed herein and a component disclosed herein in a suitable solvent to form a component-coated scaffold; b) exposing the component-coated scaffold to a surface of an appropriate-sized target particle; and c) transferring at least some of the component onto a surface of the target particle.

A method for efficiently coating target particles disclosed herein may also be performed by first forming a self-assembled scaffold using one or more components disclosed herein. The self-assembled scaffold may then be exposed to a surface of a target particle. During this exposure, the component disclosed herein is transferred from the self-assembled scaffold to the surface of the target particle in a manner which is more efficient than would otherwise be possible by exposing the component disclosed herein alone to the surface of the target particle. In other words, the presence of the self-assembled scaffold facilitates a more effective transfer of a component disclosed herein to the surface of a target particle. This methodology is general, and thus has wide applicability to a broad range of industries in which it is desired to accurately and efficiently coat target surfaces.

In an embodiment, a method for coating a surface comprises a) producing, dissolving, or suspending one or more component disclosed herein to form a self-assembled scaffold; b) exposing the self-assembled scaffold to a surface of an appropriate-sized target particle; and c) transferring at least some of the component onto a surface of the target particle.

Any mixing or loading process capable of associating a component disclosed herein onto a scaffold disclosed herein, including, without limitation, a particle or particulate lattice may be used. Non-limiting examples of mixing include dialysis, diafiltration, tangential-flow filtration, spray-drying, supercritical-fluid evaporation, precipitation, or electroprecipitation

In an embodiment, a component disclosed herein is mixed or loaded with a scaffold disclosed herein, including, without limitation, a particulate lattice, through dialysis, including, without limitation, related techniques such as dialysis, diafiltration, and tangential-flow filtration (TFF). The relative speed through and the porosity of the filtration membrane with which the mixture is dialyzed is, without limitation, capable of affecting the size and composition of the resulting coated particles which are formed. In an aspect of this embodiment, dialysis is conducted within a specific flux window, including, without limitation, a fractional volume reduction per unit time, in order to obtain coated particles which can then transfer their coatings to other surfaces. In an embodiment, a scaffold, including, without limitation, a particulate lattice need not even be present; a component disclosed herein can be condensed onto itself to form particles which are sufficiently large to accomplish the transfer of the coating material to a surface of a target particle.

In another embodiment, a component disclosed herein is mixed or loaded with a scaffold disclosed herein, including, without limitation, a particulate lattice, through condensation using a solvent/non-solvent pair. In this procedure, the desired component and scaffold, including, without limitation, a particulate lattice are mixed in the solvent and then the non-solvent is added to precipitate out the component, which then coats the scaffold. The component-coated scaffold may or may not also be precipitated out of solution. Although the procedure described herein for condensing a component disclosed herein onto a scaffold, including, without limitation, a particulate lattice have been provided, it will be apparent to one skilled in the art that there are numerous alternative methods for accomplishing the same result and the disclosed methods are not intended in any way to limit the scope of the present invention.

In another embodiment, a component disclosed herein is mixed or loaded with a scaffold disclosed herein, including, without limitation, a particulate lattice, through a spray-dried technique using any of the methods for so doing well-known to one skilled in the art to achieve the component-coated scaffold. In this process, the scaffold and/or target particles can either be included with the component in the solution or suspension, placed outside the solution or suspension and used as a target for the spray-drying process. In an aspect of this process, a separate scaffold is not incorporated, with the one or more components condensing onto themselves to form a component-coated scaffold which is capable of transferring the particles to a surface of a target particle. The concentration of a component on a scaffold or surface of the target particle can be controlled by the concentration of a component in the solution that is used to coat the particle.

A target particle may include, without limitation, a microorganism, a cell, a subcellular organelle, or a synthetic object. In aspects of this embodiment, a microorganism includes, without limitation, a virus, a bacterium, a mold, a fungi, a protozoan. In aspects of this embodiment, a synthetic particle may be the outer and/or inner surface of a prosthetic device or else a flat surface such as a diagnostic test plate.

A surface of a target particle may be any geometrical two or three-dimensional shape from nanoscale and microscale particles to complex macroscopic 3-D and even planar surfaces. In aspects of this embodiment, a surface of a target particle may be, e.g., square, rectangular, trapezoidal, pentagonal, hexagonal, heptagonal, octagonal, nonagonal, decagonal, round, oval, semicircular, or is some other two-dimensional shape.

In another embodiment, the transfer of a proanthocyanidin to a microorganism is accelerated and improved by pre-loading a proanthocyanidin onto an appropriately-sized scaffold, including, without limitation, a particulate lattice. A scaffold disclosed herein can be prepared by any of several routes, including the present rapid-diafiltration method, the method disclosed U.S. Patent Publication No. 20100221281, which is hereby incorporated by reference, or other methods, starting with the appropriate products of a fruit plant, including, without limitation, a grape-seed extract or any other proanthocyanidin-rich material. In an embodiment, a scaffold, including, without limitation, a particulate lattice is prepared or acquired and then loaded with a component capable of coating an exposed surface, and then exposed to a target surface under the appropriate conditions. The target particle is, without limitation, a microorganism such as a virus, a bacterium, a cancer cell, a mold, or a fungus, or else any other type of surface such as a sub-cellular organelle, synthetic particle. In the case of microorganisms, the product of these efforts is, without limitation, a toxoid derived from the microorganism and coated with proanthocyanidins.

In an embodiment, a scaffold including, without limitation, a particulate lattice must not bind a component disclosed herein too tightly, or else it will not be capable of transfer to the target surface. In a further embodiment, the transfer of a component from the component-coated scaffold to a surface of a target particle takes place at an enhanced rate, such that the target particles (without limitation, a virus or a bacterium or other microorganism) or any other naturally-occurring or synthetic object becomes coated much more efficiently than would otherwise be the case in the absence of the loaded particulate-lattice structure. In another embodiment, a proanthocyanidin exhibits an innate ability to bind to hydrophobic surfaces, providing, without limitation, the transfer of a proanthocyanidin much more efficiently to other surfaces such as microorganisms which can cause disease in an individual.

In an embodiment, a component disclosed herein may be deposited onto a scaffold, including, without limitation, a particulate lattice in the same step as the particulate lattice is formed. In a further embodiment, a component disclosed herein may be deposited onto a scaffold, including, without limitation, a particulate lattice in a different step as the scaffold, including, without limitation, the particulate lattice is formed.

In one embodiment, a component disclosed herein may be deposited onto a scaffold comprising a therapeutic compound. In a further embodiment, a therapeutic compound is used, without limitation, to treat a disease, cancer, an infection, a skin ailment or other syndrome suffered by an individual.

A component-coated scaffold is mixed or loaded to a surface of a scaffold by allowing a component and scaffold to incubate together for a period of time. An incubation time may be determined, without limitation, by the type and concentration of the component, the size and morphology of the scaffold, the relative concentrations and surface area of the various reagents, the temperature and pressure, and other such reaction parameters. In determining the amount of time a component disclosed herein is to be incubated with a scaffold, such time will be determinable by one of skill in the art and is based on the following factors, without limitation, the relative amounts of a component and scaffold, the available surface area of a scaffold, and/or the desired level of surface coverage.

In an embodiment, a component may be incubated to form a self-assembled scaffold or with a separate scaffold to form a component-coated scaffold for, e.g., about 1 minute to about 48 hours. In aspects of this embodiment, a component may be incubated to form a self-assembled scaffold or with a separate scaffold to form a component-coated scaffold for, e.g., at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, at least 20 minutes, at least 21 minutes, at least 22 minutes, at least 23 minutes, at least 24 minutes, at least 25 minutes, at least 26 minutes, at least 27 minutes, at least 28 minutes, at least 29 minutes, at least 30 minutes, at least 31 minutes, at least 32 minutes, at least 33 minutes, at least 34 minutes, at least 35 minutes, at least 36 minutes, at least 37 minutes, at least 38 minutes, at least 39 minutes, at least 40 minutes, at least 41 minutes, at least 42 minutes, at least 43 minutes, at least 44 minutes, at least 45 minutes, at least 46 minutes, at least 47 minutes, at least 48 minutes, at least 49 minutes, at least 50 minutes, at least 51 minutes, at least 52 minutes, at least 53 minutes, at least 54 minutes, at least 55 minutes, at least 56 minutes, at least 57 minutes, at least 58 minutes, at least 59 minutes, at least 60 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 37 hours, at least 38 hours, at least 39 hours, at least 40 hours, at least 41 hours, at least 42 hours, at least 43 hours, at least 44 hours, at least 45 hours, at least 46 hours, at least 47 hours, at least 48 hours or more.

In aspects of this embodiment, a component may be incubated to form a self-assembled scaffold or with a separate scaffold to form a component-coated scaffold for, e.g., at most 1 minute, at most 2 minutes, at most 3 minutes, at most 4 minutes, at most 5 minutes, at most 6 minutes, at most 7 minutes, at most 8 minutes, at most 9 minutes, at most 10 minutes, at most 11 minutes, at most 12 minutes, at most 13 minutes, at most 14 minutes, at most 15 minutes, at most 16 minutes, at most 17 minutes, at most 18 minutes, at most 19 minutes, at most 20 minutes, at most 21 minutes, at most 22 minutes, at most 23 minutes, at most 24 minutes, at most 25 minutes, at most 26 minutes, at most 27 minutes, at most 28 minutes, at most 29 minutes, at most 30 minutes, at most 31 minutes, at most 32 minutes, at most 33 minutes, at most 34 minutes, at most 35 minutes, at most 36 minutes, at most 37 minutes, at most 38 minutes, at most 39 minutes, at most 40 minutes, at most 41 minutes, at most 42 minutes, at most 43 minutes, at most 44 minutes, at most 45 minutes, at most 46 minutes, at most 47 minutes, at most 48 minutes, at most 49 minutes, at most 50 minutes, at most 51 minutes, at most 52 minutes, at most 53 minutes, at most 54 minutes, at most 55 minutes, at most 56 minutes, at most 57 minutes, at most 58 minutes, at most 59 minutes, at most 60 minutes, at most 1 hour, at most 2 hours, at most 3 hours, at most 4 hours, at most 5 hours, at most 6 hours, at most 7 hours, at most 8 hours, at most 9 hours, at most 10 hours, at most 11 hours, at most 12 hours, at most 13 hours, at most 14 hours, at most 15 hours, at most 16 hours, at most 17 hours, at most 18 hours, at most 19 hours, at most 20 hours, at most 21 hours, at most 22 hours, at most 23 hours, at most 24 hours, at most 25 hours, at most 26 hours, at most 27 hours, at most 28 hours, at most 29 hours, at most 30 hours, at most 31 hours, at most 32 hours, at most 33 hours, at most 34 hours, at most 35 hours, at most 36 hours, at most 37 hours, at most 38 hours, at most 39 hours, at most 40 hours, at most 41 hours, at most 42 hours, at most 43 hours, at most 44 hours, at most 45 hours, at most 46 hours, at most 47 hours, or at most 48 hours.

In aspects of this embodiment, a component may be incubated to form a self-assembled scaffold or with a separate scaffold to form a component-coated scaffold for, e.g., about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 1 minute to about 35 minutes, about 1 minute to about 40 minutes, about 1 minute to about 45 minutes, about 1 minute to about 50 minutes, about 1 minute to about 55 minutes, about 1 minute to about 60 minutes, about 5 minutes to about 10 minutes, about 5 minutes to about 15 minutes, about 5 minutes to about 20 minutes, about 5 minutes to about 25 minutes, about 5 minutes to about 30 minutes, about 5 minutes to about 35 minutes, about 5 minutes to about 40 minutes, about 5 minutes to about 45 minutes, about 5 minutes to about 50 minutes, about 5 minutes to about 55 minutes, about 5 minutes to about 60 minutes, about 10 minutes to about 15 minutes, about 10 minutes to about 20 minutes, about 10 minutes to about 25 minutes, about 10 minutes to about 30 minutes, about 10 minutes to about 35 minutes, about 10 minutes to about 40 minutes, about 10 minutes to about 45 minutes, about 10 minutes to about 50 minutes, about 10 minutes to about 55 minutes, about 10 minutes to about 60 minutes, about 15 minutes to about 20 minutes, about 15 minutes to about 25 minutes, about 15 minutes to about 30 minutes, about 15 minutes to about 35 minutes, about 15 minutes to about 40 minutes, about 15 minutes to about 45 minutes, about 15 minutes to about 50 minutes, about 15 minutes to about 55 minutes, about 15 minutes to about 60 minutes, about 30 minutes to about 60 minutes, about 1 hour to about 2 hours, about 1 hour to about 3 hours, about 1 hour to about 4 hours, about 1 hour to about 5 hours, about 1 hour to about 6 hours, about 1 hour to about 7 hours, about 1 hour to about 8 hours, about 1 hour to about 9 hours, about 1 hour to about 10 hours, about 1 hour to about 12 hours, about 1 hour to about 14 hours, about 1 hour to about 16 hours, about 1 hour to about 18 hours, about 1 hour to about 20 hours, about 1 hour to about 22 hours, about 1 hour to about 24 hours, about 1 hour to about 30 hours, about 1 hour to about 36 hours, about 1 hour to about 42 hours, about 1 hour to about 48 hours, about 2 hours to about 3 hours, about 2 hours to about 4 hours, about 2 hours to about 5 hours, about 2 hours to about 6 hours, about 2 hours to about 7 hours, about 2 hours to about 8 hours, about 2 hours to about 9 hours, about 2 hours to about 10 hours, about 2 hours to about 12 hours, about 4 hours to about 5 hours, about 4 hours to about 6 hours, about 4 hours to about 7 hours, about 4 hours to about 8 hours, about 4 hours to about 9 hours, about 4 hours to about 10 hours, about 4 hours to about 12 hours, about 6 hours to about 8 hours, about 6 hours to about 9 hours, about 6 hours to about 10 hours, about 6 hours to about 12 hours, about 6 hours to about 12 hours, about 6 hours to about 16 hours, 6 hours to about 20 hours, about 6 hours to about 24 hours, about 6 hours to about 30 hours, about 6 hours to about 36 hours, about 6 hours to about 42 hours, about 6 hours to about 48 hours, about 12 hours to about 16 hours, 12 hours to about 20 hours, about 12 hours to about 24 hours, about 12 hours to about 30 hours, about 12 hours to about 36 hours, about 12 hours to about 42 hours, about 12 hours to about 48 hours, about 24 hours to about 30 hours, about 24 hours to about 36 hours, about 24 hours to about 42 hours, or about 24 hours to about 48 hours.

A self-assembled scaffold or component-coated scaffold is exposed to a surface of a target particle by allowing a component-coated scaffold and target particle to incubate together for a period of time. An incubation time may be determined, without limitation, by the type and concentration of the self-assembled scaffold or component-coated scaffold, the size and morphology of the target particle, the relative concentrations and surface area of the various reagents, the temperature and pressure, and other such reaction parameters. In determining the amount of time a self-assembled scaffold or a component-coated scaffold disclosed herein is to be incubated with a target particle, such time will be determinable by one of skill in the art and is based on the following factors, without limitation, the relative amounts of a self-assembled scaffold and/or component-coated scaffold and target particle, the available surface area of a target particle, and/or the desired level of surface coverage.

In an embodiment, a self-assembled scaffold and/or component-coated scaffold may be incubated with a target particle for about 1 minute to about 48 hours. In aspects of this embodiment, a component may be incubated with a scaffold for, e.g., at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, at least 20 minutes, at least 21 minutes, at least 22 minutes, at least 23 minutes, at least 24 minutes, at least 25 minutes, at least 26 minutes, at least 27 minutes, at least 28 minutes, at least 29 minutes, at least 30 minutes, at least 31 minutes, at least 32 minutes, at least 33 minutes, at least 34 minutes, at least 35 minutes, at least 36 minutes, at least 37 minutes, at least 38 minutes, at least 39 minutes, at least 40 minutes, at least 41 minutes, at least 42 minutes, at least 43 minutes, at least 44 minutes, at least 45 minutes, at least 46 minutes, at least 47 minutes, at least 48 minutes, at least 49 minutes, at least 50 minutes, at least 51 minutes, at least 52 minutes, at least 53 minutes, at least 54 minutes, at least 55 minutes, at least 56 minutes, at least 57 minutes, at least 58 minutes, at least 59 minutes, at least 60 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 37 hours, at least 38 hours, at least 39 hours, at least 40 hours, at least 41 hours, at least 42 hours, at least 43 hours, at least 44 hours, at least 45 hours, at least 46 hours, at least 47 hours, at least 48 hours or more.

In aspects of this embodiment, a self-assembled scaffold and/or component-coated scaffold may be incubated with a target particle for, e.g., at most 1 minute, at most 2 minutes, at most 3 minutes, at most 4 minutes, at most 5 minutes, at most 6 minutes, at most 7 minutes, at most 8 minutes, at most 9 minutes, at most 10 minutes, at most 11 minutes, at most 12 minutes, at most 13 minutes, at most 14 minutes, at most 15 minutes, at most 16 minutes, at most 17 minutes, at most 18 minutes, at most 19 minutes, at most 20 minutes, at most 21 minutes, at most 22 minutes, at most 23 minutes, at most 24 minutes, at most 25 minutes, at most 26 minutes, at most 27 minutes, at most 28 minutes, at most 29 minutes, at most 30 minutes, at most 31 minutes, at most 32 minutes, at most 33 minutes, at most 34 minutes, at most 35 minutes, at most 36 minutes, at most 37 minutes, at most 38 minutes, at most 39 minutes, at most 40 minutes, at most 41 minutes, at most 42 minutes, at most 43 minutes, at most 44 minutes, at most 45 minutes, at most 46 minutes, at most 47 minutes, at most 48 minutes, at most 49 minutes, at most 50 minutes, at most 51 minutes, at most 52 minutes, at most 53 minutes, at most 54 minutes, at most 55 minutes, at most 56 minutes, at most 57 minutes, at most 58 minutes, at most 59 minutes, at most 60 minutes, at most 1 hour, at most 2 hours, at most 3 hours, at most 4 hours, at most 5 hours, at most 6 hours, at most 7 hours, at most 8 hours, at most 9 hours, at most 10 hours, at most 11 hours, at most 12 hours, at most 13 hours, at most 14 hours, at most 15 hours, at most 16 hours, at most 17 hours, at most 18 hours, at most 19 hours, at most 20 hours, at most 21 hours, at most 22 hours, at most 23 hours, at most 24 hours, at most 25 hours, at most 26 hours, at most 27 hours, at most 28 hours, at most 29 hours, at most 30 hours, at most 31 hours, at most 32 hours, at most 33 hours, at most 34 hours, at most 35 hours, at most 36 hours, at most 37 hours, at most 38 hours, at most 39 hours, at most 40 hours, at most 41 hours, at most 42 hours, at most 43 hours, at most 44 hours, at most 45 hours, at most 46 hours, at most 47 hours, or at most 48 hours.

In aspects of this embodiment, a self-assembled scaffold and/or component-coated scaffold may be incubated with a target particle for, e.g., about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 1 minute to about 35 minutes, about 1 minute to about 40 minutes, about 1 minute to about 45 minutes, about 1 minute to about 50 minutes, about 1 minute to about 55 minutes, about 1 minute to about 60 minutes, about 5 minutes to about 10 minutes, about 5 minutes to about 15 minutes, about 5 minutes to about 20 minutes, about 5 minutes to about 25 minutes, about 5 minutes to about 30 minutes, about 5 minutes to about 35 minutes, about 5 minutes to about 40 minutes, about 5 minutes to about 45 minutes, about 5 minutes to about 50 minutes, about 5 minutes to about 55 minutes, about 5 minutes to about 60 minutes, about 10 minutes to about 15 minutes, about 10 minutes to about 20 minutes, about 10 minutes to about 25 minutes, about 10 minutes to about 30 minutes, about 10 minutes to about 35 minutes, about 10 minutes to about 40 minutes, about 10 minutes to about 45 minutes, about 10 minutes to about 50 minutes, about 10 minutes to about 55 minutes, about 10 minutes to about 60 minutes, about 15 minutes to about 20 minutes, about 15 minutes to about 25 minutes, about 15 minutes to about 30 minutes, about 15 minutes to about 35 minutes, about 15 minutes to about 40 minutes, about 15 minutes to about 45 minutes, about 15 minutes to about 50 minutes, about 15 minutes to about 55 minutes, about 15 minutes to about 60 minutes, about 30 minutes to about 60 minutes, about 1 hour to about 2 hours, about 1 hour to about 3 hours, about 1 hour to about 4 hours, about 1 hour to about 5 hours, about 1 hour to about 6 hours, about 1 hour to about 7 hours, about 1 hour to about 8 hours, about 1 hour to about 9 hours, about 1 hour to about 10 hours, about 1 hour to about 12 hours, about 1 hour to about 14 hours, about 1 hour to about 16 hours, about 1 hour to about 18 hours, about 1 hour to about 20 hours, about 1 hour to about 22 hours, about 1 hour to about 24 hours, about 1 hour to about 30 hours, about 1 hour to about 36 hours, about 1 hour to about 42 hours, about 1 hour to about 48 hours, about 2 hours to about 3 hours, about 2 hours to about 4 hours, about 2 hours to about 5 hours, about 2 hours to about 6 hours, about 2 hours to about 7 hours, about 2 hours to about 8 hours, about 2 hours to about 9 hours, about 2 hours to about 10 hours, about 2 hours to about 12 hours, about 4 hours to about 5 hours, about 4 hours to about 6 hours, about 4 hours to about 7 hours, about 4 hours to about 8 hours, about 4 hours to about 9 hours, about 4 hours to about 10 hours, about 4 hours to about 12 hours, about 6 hours to about 8 hours, about 6 hours to about 9 hours, about 6 hours to about 10 hours, about 6 hours to about 12 hours, about 6 hours to about 12 hours, about 6 hours to about 16 hours, 6 hours to about 20 hours, about 6 hours to about 24 hours, about 6 hours to about 30 hours, about 6 hours to about 36 hours, about 6 hours to about 42 hours, about 6 hours to about 48 hours, about 12 hours to about 16 hours, 12 hours to about 20 hours, about 12 hours to about 24 hours, about 12 hours to about 30 hours, about 12 hours to about 36 hours, about 12 hours to about 42 hours, about 12 hours to about 48 hours, about 24 hours to about 30 hours, about 24 hours to about 36 hours, about 24 hours to about 42 hours, or about 24 hours to about 48 hours.

In an embodiment, a component disclosed herein covers or coats the surface of a separate scaffold, including, without limitation, a surface of a particulate lattice. In aspects of this embodiment, the component disclosed herein covers or coats, e.g., at least 1%, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% of a surface of a separate scaffold, including, without limitation, a surface of a particulate lattice. In other aspects of this embodiment, the component disclosed herein covers or coats, e.g., at most 1%, at most 2%, at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95% of a surface of a separate scaffold, including, without limitation, a surface of a particulate lattice.

In other aspects of this embodiment, the component disclosed herein covers or coats, e.g., about 1% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% of a separate surface of a scaffold, including, without limitation, a surface of a particulate lattice.

In an embodiment, a component disclosed herein covers or coats a surface of a target particle including, without limitation, a surface of a microorganism, a cell, a subcellular organelle, or a synthetic object. In aspects of this embodiment, the component disclosed herein covers or coats, e.g., at least 1%, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% of a surface of a target particle including, without limitation, a surface of a microorganism, a cell, a subcellular organelle, or a synthetic object. In other aspects of this embodiment, the component disclosed herein covers or coats, e.g., at most 1%, at most 2%, at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90% or at most 95% of a surface of a target particle including, without limitation, a surface of a microorganism, a cell, a subcellular organelle, or a synthetic object.

In other aspects of this embodiment, the component disclosed herein covers or coats, e.g., about 1% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% of a surface of a target particle including, without limitation, a surface of a microorganism, a cell, a subcellular organelle, or a synthetic object.

In an embodiment, a component disclosed herein and/or a scaffold disclosed herein and/or a self-assembled scaffold disclosed herein and/or a component-coated scaffold disclosed herein and/or a component-coated target particle disclosed herein may be administered to an individual as a pharmaceutical composition. In an aspect of this embodiment, a pharmaceutical composition comprises a component-coated microorganism, such as, e.g., a proanthocyanidin-coated microorganism, such as, e.g., a proanthocyanidin-coated virus, a proanthocyanidin-coated toxoid, a proanthocyanidin-coated bacterium, a proanthocyanidin-coated mold, a proanthocyanidin-coated fungus, a proanthocyanidin-coated protozoan.

A pharmaceutical composition disclosed herein is capable of reducing the severity of a disease, cancer, an infection, a skin ailment or other syndrome. In aspects of this embodiment, a therapeutic compound may be capable of reducing the severity of a disease, cancer, infection, skin ailment or other syndrome of an individual suffering from a disease, cancer, infection, skin ailment or other syndrome by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In another aspects of this embodiment, a therapeutic compound capable of reducing the severity of a disease, cancer, infection, skin ailment or other syndrome in an individual suffering from a disease, cancer, infection, skin ailment or other syndrome by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

The final concentration of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein in a pharmaceutical composition disclosed herein may be of any concentration desired. In an aspect of this embodiment, the final concentration of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein in a pharmaceutical composition may be a therapeutically effective amount.

In other aspects of this embodiment, the final concentration of a component disclosed herein and/or a scaffold disclosed herein and/or a self-assembled scaffold disclosed herein and/or a component-coated scaffold disclosed herein and/or a component-coated target particle disclosed herein in a pharmaceutical composition disclosed herein may be, e.g., at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL, at least 500 mg/mL, at least 700 mg/mL, at least 1,000 mg/mL, or at least 1,200 mg/mL. In other aspects of this embodiment, the final concentration of a component disclosed herein and/or a scaffold disclosed herein and/or a self-assembled scaffold disclosed herein and/or a component-coated scaffold disclosed herein and/or a component-coated target particle disclosed herein in a pharmaceutical composition disclosed herein may be, e.g., at most 1,000 mg/mL, at most 1,100 mg/mL, at most 1,200 mg/mL, at most 1,300 mg/mL, at most 1,400 mg/mL, at most 1,500 mg/mL, at most 2,000 mg/mL, at most 2,000 mg/mL, or at most 3,000 mg/mL.

In other aspects of this embodiment, the final concentration of a component disclosed herein and/or a scaffold disclosed herein and/or a self-assembled scaffold disclosed herein and/or a component-coated scaffold disclosed herein and/or a component-coated target particle disclosed herein in a pharmaceutical composition disclosed herein may be in a range of, e.g., about 0.00001 mg/mL to about 3,000 mg/mL, about 0.0001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/mL to about 100 mg/mL.

A pharmaceutical composition disclosed herein may comprise, in addition to a component disclosed herein and/or a scaffold disclosed herein and/or a self-assembled scaffold disclosed herein and/or a component-coated scaffold disclosed herein and/or a component-coated target particle disclosed herein, one or more therapeutic compounds and one or more pharmacologically-acceptable carriers. A pharmaceutical composition disclosed herein may optionally include a pharmaceutically-acceptable carrier that facilitates processing of an active ingredient into pharmaceutically-acceptable compositions. As used herein, the term “pharmacologically-acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary or excipient.” Such a carrier generally is mixed with an active compound or permitted to dilute or enclose the active compound and can be a solid, semi-solid, or liquid agent. It is understood that the active ingredients can be soluble or can be delivered as a suspension in the desired carrier or diluent. Any of a variety of pharmaceutically-acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. The selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7th ed. 1999); REMINGTON: THE SCIENCE AND PRACTICE OF PHARMACY (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10th ed. 2001); and Handbook of Pharmaceutical Excipients (Raymond C. Rowe et al., APhA Publications, 4th edition 2003). These protocols are routine procedures and any modifications are well within the scope of one skilled in the art and from the teaching herein.

In an embodiment a carrier for a component-coated target particle including, without limitation, a microorganism, a cell, a subcellular organelle, or a synthetic object can include, without limitation, neat water and buffered aqueous solutions such as phosphate-buffered saline (PBS), or any other such solvent systems which are known to those skilled in the art.

A pharmaceutical composition disclosed herein can optionally include, without limitation, other pharmaceutically acceptable components (or pharmaceutical components), including, without limitation, active pharmaceutical ingredients, buffers, preservatives, tonicity adjusters, salts, antioxidants, osmolality adjusting agents, cryoprotectants, mold-release agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition.

A pharmaceutical composition disclosed herein may be formulated for either local or systemic delivery using topical, enteral, or parenteral routes of administration. Additionally, a component disclosed herein and/or a scaffold disclosed herein and/or a self-assembled scaffold disclosed herein and/or a component-coated scaffold disclosed herein and/or a component-coated target particle disclosed herein may be formulated by itself in a pharmaceutical composition, or may be formulated together with one or more other components disclosed herein and/or scaffolds disclosed herein and/or self-assembled scaffolds disclosed herein and/or component-coated scaffolds disclosed herein and/or component-coated target particles disclosed herein and/or other therapeutic compounds in a single pharmaceutical composition.

A pharmaceutical composition disclosed herein may comprise a pharmaceutically-acceptable adjuvant in an amount sufficient to mix with a solution disclosed herein or an emulsion disclosed herein. In other aspects of this embodiment, a pharmaceutical composition disclosed herein may comprise an adjuvant in an amount of, e.g., at least 10% (v/v), at least 20% (v/v), at least 30% (v/v), at least 35% (v/v), at least 40% (v/v), at least 45% (v/v), at least 50% (v/v), at least 55% (v/v), at least 60% (v/v), at least 65% (v/v), at least 70% (v/v), at least 75% (v/v), at least 80% (v/v), at least 85% (v/v), at least 90% (v/v), at least 95% (v/v), or at least 99% (v/v). In other aspects of this embodiment, a pharmaceutical composition disclosed herein may comprise an adjuvant in an amount in a range of, e.g., about 30% (v/v) to about 99% (v/v), about 35% (v/v) to about 99% (v/v), about 40% (v/v) to about 99% (v/v), about 45% (v/v) to about 99% (v/v), about 50% (v/v) to about 99% (v/v), about 30% (v/v) to about 98% (v/v), about 35% (v/v) to about 98% (v/v), about 40% (v/v) to about 98% (v/v), about 45% (v/v) to about 98% (v/v), about 50% (v/v) to about 98% (v/v), about 30% (v/v) to about 95% (v/v), about 35% (v/v) to about 95% (v/v), about 40% (v/v) to about 95% (v/v), about 45% (v/v) to about 95% (v/v), or about 50% (v/v) to about 95% (v/v). In yet other aspects of this embodiment, a pharmaceutical composition disclosed herein may comprise an adjuvant in an amount in a range of, e.g., about 70% (v/v) to about 97% (v/v), about 75% (v/v) to about 97% (v/v), about 80% (v/v) to about 97% (v/v), about 85% (v/v) to about 97% (v/v), about 88% (v/v) to about 97% (v/v), about 89% (v/v) to about 97% (v/v), about 90% (v/v) to about 97% (v/v), about 75% (v/v) to about 96% (v/v), about 80% (v/v) to about 96% (v/v), about 85% (v/v) to about 96% (v/v), about 88% (v/v) to about 96% (v/v), about 89% (v/v) to about 96% (v/v), about 90% (v/v) to about 96% (v/v), about 75% (v/v) to about 93% (v/v), about 80% (v/v) to about 93% (v/v), about 85% (v/v) to about 93% (v/v), about 88% (v/v) to about 93% (v/v), about 89% (v/v) to about 93% (v/v), or about 90% (v/v) to about 93% (v/v).

In an embodiment, the immune response generated by a pharmaceutical composition disclosed herein can be augmented through the use of an adjuvant, including, without limitation, alum (aluminum hydroxide), widely available from laboratory supply companies such as Pierce (Rockford, Ill.), and keyhole limpet hemocyanin (KLH), available from Stellar Biotech (Port Hueneme, Calif.), oil emulsions, toxins, aluminum salts, including without limitation, aluminum hydroxide along with others.

In one embodiment, an adjuvant may be a pharmaceutically-acceptable lipid. A lipid may be broadly defined as a hydrophobic or amphiphilic small molecule. The amphiphilic nature of some lipids allows them to form structures such as vesicles, liposomes, or membranes in an aqueous environment. Non-limiting examples of lipids include fatty acids, glycerolipids (such as monoglycerides, diglycerides, and triglycerides), phospholipids, sphingolipids, sterol lipids, prenol lipids, saccharolipids, and polyketides. A pharmaceutical composition disclosed herein may comprise a lipid such as, e.g. an oil, an oil-based liquid, a fat, a fatty acid, a partially hydrolyzed fatty acid, a wax, a fatty acid ester, a fatty acid salt, a fatty alcohol, a glyceride (mono-, di- or tri-glyceride), a phospholipids, a glycol ester, a sucrose ester, a glycerol oleate derivative, a medium chain triglyceride, a partially hydrolyzed triglyceride, or a mixture thereof.

A pharmaceutical composition disclosed herein can be formulated as a controlled release formulation including a sustained release formulation and an extended release formulation. A sustained release formulation refers to the release of a component disclosed herein and/or a scaffold disclosed herein and/or a self-assembled scaffold disclosed herein and/or a component-coated scaffold disclosed herein and/or a component-coated target particle disclosed herein over a period of about seven days or more. In aspects of this embodiment, a sustained release formulation releases a component disclosed herein and/or a scaffold disclosed herein and/or a self-assembled scaffold disclosed herein and/or a component-coated scaffold disclosed herein and/or a component-coated target particle disclosed herein with substantially zero-order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release formulation releases a component disclosed herein and/or a scaffold disclosed herein and/or a self-assembled scaffold disclosed herein and/or a component-coated scaffold disclosed herein and/or a component-coated target particle disclosed herein with substantially zero-order release kinetics over a period of, e.g., at least 7 days after administration, at least 15 days after administration, at least 30 days after administration, at least 45 days after administration, at least 60 days after administration, at least 75 days after administration, or at least 90 days after administration.

In aspects of this embodiment, a sustained release formulation releases a component disclosed herein and/or a scaffold disclosed herein and/or a self-assembled scaffold disclosed herein and/or a component-coated scaffold disclosed herein and/or a component-coated target particle disclosed herein with substantially first-order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release formulation releases a component disclosed herein and/or a scaffold disclosed herein and/or a self-assembled scaffold disclosed herein and/or a component-coated scaffold disclosed herein and/or a component-coated target particle disclosed herein with substantially first-order release kinetics over a period of, e.g., at least 7 days after administration, at least 15 days after administration, at least 30 days after administration, at least 45 days after administration, at least 60 days after administration, at least 75 days after administration, or at least 90 days after administration.

An extended release formulation refers to the release of a component disclosed herein and/or a scaffold disclosed herein and/or a self-assembled scaffold disclosed herein and/or a component-coated scaffold disclosed herein and/or a component-coated target particle disclosed herein over a period of time of less than seven days. In aspects of this embodiment, an extended release formulation releases a component disclosed herein and/or a scaffold disclosed herein and/or a self-assembled scaffold disclosed herein and/or a component-coated scaffold disclosed herein and/or a component-coated target particle disclosed herein with substantially zero-order release kinetics over a period of, e.g., about 1 day after administration, about 2 days after administration, about 3 days after administration, about 4 days after administration, about 5 days after administration, or about 6 days after administration. In other aspects of this embodiment, an extended release formulation releases a component disclosed herein and/or a scaffold disclosed herein and/or a self-assembled scaffold disclosed herein and/or a component-coated scaffold disclosed herein and/or a component-coated target particle disclosed herein with substantially zero-order release kinetics over a period of, e.g., at most 1 day after administration, at most 2 days after administration, at most 3 days after administration, at most 4 days after administration, at most 5 days after administration, or at most 6 days after administration.

In aspects of this embodiment, an extended release formulation releases a component disclosed herein and/or a scaffold disclosed herein and/or a self-assembled scaffold disclosed herein and/or a component-coated scaffold disclosed herein and/or a component-coated target particle disclosed herein with substantially first-order release kinetics over a period of, e.g., about 1 day after administration, about 2 days after administration, about 3 days after administration, about 4 days after administration, about 5 days after administration, or about 6 days after administration. In other aspects of this embodiment, an extended release formulation releases a component disclosed herein and/or a scaffold disclosed herein and/or a self-assembled scaffold disclosed herein and/or a component-coated scaffold disclosed herein and/or a component-coated target particle disclosed herein with substantially first-order release kinetics over a period of, e.g., at most 1 day after administration, at most 2 days after administration, at most 3 days after administration, at most 4 days after administration, at most 5 days after administration, or at most 6 days after administration.

The formulations and methods disclosed herein are intended to be illustrative and not limiting. It will be appreciated by those skilled in the art that alternative formulations, methods, and products can readily be devised using the methods and materials of the present invention.

In an embodiment, a pharmaceutical composition comprises one or more components disclosed herein and/or scaffolds disclosed herein and/or self-assembled scaffolds disclosed herein and/or component-coated scaffolds disclosed herein and/or component-coated target particles disclosed herein, with each a therapeutically effective amount of a component disclosed herein and/or a scaffold disclosed herein and/or a self-assembled scaffold disclosed herein and/or a component-coated scaffold disclosed herein and/or a component-coated target particle disclosed herein present in an amount of, e.g., at least 0.01%, at least 0.02%, at least 0.05%, at least 0.075%, at least 0.1%, at least 0.2%, at least 0.3%, at least 0.4%, at least 0.5%, at least 0.6%, at least 0.7%, at least 0.8%, at least 0.9%, at least 1%, at least 1.5%, at least 1.75%, at least 2%, at least 2.25%, at least 2.5%, at least 2.75%, at least 3%, at least 3.25%, at least 3.5%, at least 3.75%, at least 4%, at least 4.25%, at least 4.5%, at least 4.75%, at least 5%, at least 5.25%, at least 5.5%, at least 5.75%, at least 6%, 6.25%, at least 6.5%, at least 6.75%, at least 7%, at least 7.25%, at least 7.5%, at least 7.75%, at least 8%, at least 8.25%, at least 8.5%, at least 8.75%, at least 9%, at least 9.25%, at least 9.5%, at least 9.75%, at least 10%, at least 10.25%, at least 10.5%, at least 10.75%, at least 11%, at least 11.25%, at least 11.5%, at least 11.75%, at least 12%, at least 12.25%, at least 12.5%, at least 12.75%, at least 13%, at least 13.25%, at least 13.5%, at least 13.75%, at least 14%, at least 14.25%, at least 14.5%, at least 14.75%, at least 15%, at least 15.25%, at least 15.5%, at least 15.75%, at least 16%, at least 16.25%, at least 16.5%, at least 16.75%, at least 17%, at least 17.25%, at least 17.5%, at least 17.75%, at least 18%, at least 18.25%, at least 18.5%, at least 18.75%, at least 19%, at least 19.25%, at least 19.5%, at least 19.75%, at least 20%, at least 20.25%, at least 20.5%, at least 20.75%, at least 21%, at least 21.25%, at least 21.5%, at least 21.75%, at least 22%, at least 22.25%, at least 22.5%, at least 22.75%, at least 23%, at least 23.25%, at least 23.5%, at least 23.75%, at least 24%, at least 24.25%, at least 24.5%, at least 24.75%, at least 25%, at least 26%, at least 27%, at least 28%, at least 29%, at least 30%, at least 31%, at least 32%, at least 33%, at least 34%, at least 35%, at least 40%, or more by weight of the pharmaceutical composition.

In an embodiment, a pharmaceutical composition comprises one or more components disclosed herein and/or scaffolds disclosed herein and/or self-assembled scaffolds disclosed herein and/or component-coated scaffolds disclosed herein and/or component-coated target particles disclosed herein, with each a therapeutically effective amount of a component disclosed herein and/or a scaffold disclosed herein and/or a self-assembled scaffold disclosed herein and/or a component-coated scaffold disclosed herein and/or a component-coated target particle disclosed herein present in an amount of, e.g., at most 0.01%, at most 0.02%, at most 0.05%, at most 0.075%, at most 0.1%, at most 0.2%, at most 0.3%, at most 0.4%, at most 0.5%, at most 0.6%, at most 0.7%, at most 0.8%, at most 0.9%, at most 1%, at most 1.5%, at most 1.75%, at most 2%, at most 2.25%, at most 2.5%, at most 2.75%, at most 3%, at most 3.25%, at most 3.5%, at most 3.75%, at most 4%, at most 4.25%, at most 4.5%, at most 4.75%, at most 5%, at most 5.25%, at most 5.5%, at most 5.75%, at most 6%, 6.25%, at most 6.5%, at most 6.75%, at most 7%, at most 7.25%, at most 7.5%, at most 7.75%, at most 8%, at most 8.25%, at most 8.5%, at most 8.75%, at most 9%, at most 9.25%, at most 9.5%, at most 9.75%, at most 10%, at most 10.25%, at most 10.5%, at most 10.75%, at most 11%, at most 11.25%, at most 11.5%, at most 11.75%, at most 12%, at most 12.25%, at most 12.5%, at most 12.75%, at most 13%, at most 13.25%, at most 13.5%, at most 13.75%, at most 14%, at most 14.25%, at most 14.5%, at most 14.75%, at most 15%, at most 15.25%, at most 15.5%, at most 15.75%, at most 16%, at most 16.25%, at most 16.5%, at most 16.75%, at most 17%, at most 17.25%, at most 17.5%, at most 17.75%, at most 18%, at most 18.25%, at most 18.5%, at most 18.75%, at most 19%, at most 19.25%, at most 19.5%, at most 19.75%, at most 20%, at most 20.25%, at most 20.5%, at most 20.75%, at most 21%, at most 21.25%, at most 21.5%, at most 21.75%, at most 22%, at most 22.25%, at most 22.5%, at most 22.75%, at most 23%, at most 23.25%, at most 23.5%, at most 23.75%, at most 24%, at most 24.25%, at most 24.5%, at most 24.75%, at most 25%, at most 26%, at most 27%, at most 28%, at most 29%, at most 30%, at most 31%, at most 32%, at most 33%, at most 34%, at most 35%, at most 40%, or more by weight of the pharmaceutical composition.

In an embodiment, a pharmaceutical composition comprises one or more components disclosed herein and/or scaffolds disclosed herein and/or self-assembled scaffolds disclosed herein and/or component-coated scaffolds disclosed herein and/or component-coated target particles disclosed herein, with each a therapeutically effective amount of a component disclosed herein and/or a scaffold disclosed herein and/or a self-assembled scaffold disclosed herein and/or a component-coated scaffold disclosed herein and/or a component-coated target particle disclosed herein present in an amount of, e.g., about 0.1% (w/v) to about 40%, or alternatively at about 0.01% to about 25%, about 0.02% to about 25%, about 0.05% to about 25%, about 0.075% to about 25%, about 0.2% to about 25%, about 0.3% to about 25%, about 0.4% to about 25%, about 0.5% to about 25%, about 0.6% to about 25%, about 0.7% to about 25%, about 0.8% to about 25%, about 0.9% to about 25%, about 1% to about 25%, about 1.5% to about 25%, about 1.75% to about 25%, about 2% to about 25%, about 2.25% to about 25%, about 2.5% to about 25%, about 2.75% to about 25%, about 3% to about 25%, about 3.25% to about 25%, about 3.5% to about 25%, about 3.75% to about 25%, about 4% to about 25%, about 4.25% to about 25%, about 4.5% to about 25%, about 4.75% to about 25%, about 5% to about 25%, about 5.25% to about 25%, about 5.5% to about 25%, about 5.75% to about 25%, about 6% to about 25%, about 6.25% to about 25%, about 6.5% to about 25%, about 6.75% to about 25%, about 7% to about 25%, about 7.25% to about 25%, about 7.5% to about 25%, about 7.75% to about 25%, about 8% to about 25%, about 8.25% to about 25%, about 8.5% to about 25%, about 8.75% to about 25%, about 9% to about 25%, about 9.25% to about 25%, about 9.5% to about 25%, about 9.75% to about 25%, about 10% to about 25%, about 10.25% to about 25%, about 10.5% to about 25%, about 10.75% to about 25%, about 11% to about 25%, about 11.25% to about 25%, about 11.5% to about 25%, about 11.75% to about 25%, about 12% to about 25%, about 12.25% to about 25%, about 12.5% to about 25%, about 12.75% to about 25%, about 13% to about 25%, about 13.25% to about 25%, about 13.5% to about 25%, about 13.75% to about 25%, about 14% to about 25%, about 14.25% to about 25%, about 14.5% to about 25%, about 14.75% to about 25%, about 15% to about 25%, about 15.25% to about 25%, about 15.5% to about 25%, about 15.75% to about 25%, about 16% to about 25%, about 16.25% to about 25%, about 16.5% to about 25%, about 16.75% to about 25%, about 17% to about 25%, about 17.25% to about 25%, about 17.5% to about 25%, about 17.75% to about 25%, about 18% to about 25%, about 18.25% to about 25%, about 18.5% to about 25%, about 18.75% to about 25%, about 19% to about 25%, about 19.25% to about 25%, about 19.5% to about 25%, about 19.75% to about 25%, about 20% to about 25%, about 20.25% to about 25%, about 20.5% to about 25%, about 20.75% to about 25%, about 5% to about 20%, about 6% to about 20%, about 7% to about 20%, about 8% to about 20%, about 9% to about 20%, about 10% to about 20%, about 11% to about 20%, about 12% to about 20%, about 13%, about to about 20%, about 14% to about 20%, about 15% to about 20%, about 5% to about 15%, about 6% to about 15%, about 7% to about 15%, about 8% to about 15%, about 9% to about 15%, about 10% to about 15%, about 11% to about 15%, about 12% to about 15%, about 13%, about to about 15%, about 14% to about 15% (w/v).

In an embodiment, a formulation is considered stable when a component disclosed herein and/or a scaffold disclosed herein and/or a self-assembled scaffold disclosed herein and/or a component-coated scaffold disclosed herein and/or a component-coated target particle disclosed herein in the formulation (1) retains its physical stability, (2) retains its chemical stability and/or (3) retains it biological activity. In an embodiment, a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be said to “retain its physical stability” in a formulation if, for example, without limitation, it shows no signs of aggregation, precipitation and/or denaturation upon visual examination of color and/or clarity, or as measured by static or dynamic light scattering or by size exclusion chromatography (SEC) or electrophoresis, such as with reference to turbidity or aggregate formation.

In an embodiment, a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be said to “retain its chemical stability” in a formulation, if, for example, without limitation, the chemical stability at a given time is such that there is no significant modification of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein by bond formation or cleavage resulting in a new chemical entity. In a further embodiment, chemical stability can be assessed by detecting and quantifying chemically altered forms of the therapeutic composition. Chemical alteration may involve, example, without limitation, size modification (e.g. clipping) which can be evaluated using size-exclusion chromatography, SDS-PAGE and/or matrix-assisted laser desorption ionization/time-of-flight mass spectrometry (MALDI-TOF MS). Other types of chemical alteration include, for example, without limitation, charge alteration (e.g. occurring in polypeptides as a result of deamidation), which can be evaluated by ion-exchange chromatography, for example. Oxidation is another commonly seen chemical modification, as is racemization or another type of rearrangement, amidation, and other such modifications well-known to those skilled in the art.

Aspects of the present specification disclose a method of treating an individual suffering from a disease, an infection, a cancer, a skin ailment, or other syndrome. A method of treatment disclosed herein comparing the step of administering a pharmaceutical composition disclosed herein. In aspects of this embodiment, a pharmaceutical composition comprises one or more components disclosed herein and/or scaffolds disclosed herein and/or self-assembled scaffolds disclosed herein and/or component-coated scaffolds disclosed herein and/or component-coated target particles disclosed herein. In other aspect of this embodiment, a pharmaceutical composition comprises one or more component-coated microorganism, such as, e.g., a proanthocyanidin-coated microorganism, such as, e.g., a proanthocyanidin-coated virus, a proanthocyanidin-coated toxoid, a proanthocyanidin-coated bacterium, a proanthocyanidin-coated mold, a proanthocyanidin-coated fungus, and/or a proanthocyanidin-coated protozoan.

A disease disclosed herein includes, without limitation, a multiple sclerosis, a rheumatoid arthritis, and a system lupus erthematosis. An infection disclosed herein includes, without limitation, a viral infection, a bacterial infection or a parasitic infection. In aspects of this embodiment, a disease or infection may be caused by Adenoviridae, Arenaviridae, Bunyaviridae, Caliciviridae, Coronaviridae, Filoviridae, Flaviviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, and/or Togaviridae.

A cancer disclosed herein includes, without limitation, Acute Lymphoblastic Leukemia; Acute Myeloid Leukemia; Karposi Sarcoma, Appendix Cancer; Adrenocortical Carcinoma; Anal Cancer; Basal Cell Carcinoma; Bladder Cancer; Blood Cancers Treatment, Brain Tumor, Adult; Brain Tumor, Brain Stem Glioma, Childhood; Brain Tumor, Cerebellar Astrocytoma, Childhood; Brain Tumor, Cerebral Astrocytoma, Childhood; Brain Tumor, Ependymoma, Childhood; Brain Tumor, Childhood (Other); Breast Cancer; Breast Cancer, Male; Carcinoid Tumor; Gastrointestinal, Carcinoma of Unknown Primary; Cervical Cancer; Colon Cancer; Endometrial Cancer; Esophageal Cancer; Extrahepatic Bile Duct Cancer; Ewings Family of Tumors (PNET); Extracranial Germ Cell Tumor, Childhood; Eye Cancer; Intraocular Melanoma; Gallbladder Cancer; Gastric Cancer (Stomach); Germ Cell Tumor; Extragonadal Gestational Trophoblastic Tumor; Head and Neck Cancer; Hypopharyngeal Cancer; Islet Cell Carcinoma; Kidney Cancer (renal cell cancer); Laryngeal Cancer; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic, Leukemia; Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoma; AIDS-Related Lymphoma; Central Nervous System (Primary); Lymphoma, Cutaneous T-Cell; Lymphoma, Hodgkin's Disease, Adult; Lymphoma, Hodgkin's Disease, Childhood; Lymphoma, Non-Hodgkin's Disease, Adult; Lymphoma, Non-Hodgkin's Disease, Childhood; Malignant Mesothelioma; Melanoma Merkel Cell Carcinoma; Metasatic Squamous Neck Cancer with Occult Primary; Multiple Myeloma and Other Plasma Cell Neoplasms; Mycosis Fungoides; Myelodysplastic Syndrome; Myeloproliferative Disorders; Nasopharyngeal Cancer; Neuroblastoma; Oral Cancer; Oropharyngeal Cancer; Osteosarcoma; Ovarian Epithelial Cancer; Ovarian Germ Cell Tumor; Pancreatic Cancer, Exocrine; Pancreatic Cancer, Islet Cell Carcinoma; Paranasal Sinus and Nasal Cavity Cancer; Parathyroid Cancer; Penile Cancer; Pituitary Cancer; Plasma Cell Neoplasm; Prostate Cancer; Rhabdomyosarcoma, Childhood; Rectal Cancer; Renal Cell Cancer (cancer of the kidney); Renal Pelvis and Ureter, Transitional Cell; Salivary Gland Cancer; Sezary Syndrome; Skin Cancer; Skin Cancer, Cutaneous T-Cell Lymphoma; Skin Cancer, Kaposi's Sarcoma; Skin Cancer, Melanoma; Small Intestine Cancer; Soft Tissue Sarcoma, Adult; Soft Tissue Sarcoma, Child; Stomach Cancer; Testicular Cancer; Thymoma, Malignant; Thyroid Cancer; Urethral Cancer; Uterine Cancer, Sarcoma Unusual Cancer of Childhood; Vaginal Cancer; Vulvar Cancer; or Wilms' Tumor.

A component disclosed herein and/or a scaffold disclosed herein and/or a self-assembled scaffold disclosed herein and/or a component-coated scaffold disclosed herein and/or a component-coated target particle disclosed herein or a pharmaceutical composition disclosed herein may be administered to an individual. An individual may be without limitation, an animal. As animal may be, without limitation, a mammal, a reptile, a bird, a fish, a marsupial or other animal. A mammal may be without limitation, a human, a cat, a dog, a cow, a horse, a goat, a sheep, a pig or other domestic or non-domesticated animal.

In an embodiment, a component disclosed herein and/or a scaffold disclosed herein and/or a self-assembled scaffold disclosed herein and/or a component-coated scaffold disclosed herein and/or a component-coated target particle disclosed herein or a pharmaceutical composition disclosed herein is administered to an individual for a period of time followed by no administration to the individual during a separate period of time. In aspects of this embodiment, a period of administration of a component disclosed herein and/or a scaffold disclosed herein and/or a self-assembled scaffold disclosed herein and/or a component-coated scaffold disclosed herein and/or a component-coated target particle disclosed herein or a pharmaceutical composition disclosed herein may be for, e.g., 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, an individual administered a component disclosed herein and/or a scaffold disclosed herein and/or a self-assembled scaffold disclosed herein and/or a component-coated scaffold disclosed herein and/or a component-coated target particle disclosed herein or a pharmaceutical composition disclosed herein has the administration stopped for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more followed by administration of a component disclosed herein and/or a scaffold disclosed herein and/or a self-assembled scaffold disclosed herein and/or a component-coated scaffold disclosed herein and/or a component-coated target particle disclosed herein or a pharmaceutical composition disclosed herein to the individual.

Various routes of administration can be useful for administering a pharmaceutical composition disclosed herein. A pharmaceutical composition may be administered to an individual by any of a variety of means depending, e.g., on the specific pharmaceutical composition used, the specific component disclosed herein and/or scaffold disclosed herein and/or self-assembled scaffold disclosed herein and/or component-coated scaffold disclosed herein and/or component-coated target particle disclosed herein, and the history, risk factors and symptoms of the individual. As such, a pharmaceutical composition disclosed herein can be administered to an individual by any enteral, parenteral or topical routes, including, without limitation, oral, inhalation, intravenous, subcutaneous, intramuscular, sublingual, rectal, transdermal, vaginal, intranasal, through eye drops, through ear drops, insufflation, depot, implantable piston, osmotic, diffusion pump, cannulae, diffusion-limiting gel, sponge or other device capable of administering the composition to an individual. Such routes disclosed herein include both local and systemic delivery of a pharmaceutical composition disclosed herein.

Pharmaceutical compositions comprising either a single a component disclosed herein and/or a scaffold disclosed herein and/or a self-assembled scaffold disclosed herein and/or a component-coated scaffold disclosed herein and/or a component-coated target particle disclosed herein, or two or more components disclosed herein and/or scaffolds disclosed herein and/or self-assembled scaffolds disclosed herein and/or component-coated scaffolds disclosed herein and/or component-coated target particles disclosed herein may be prepared according to any method known to the art for the manufacture of pharmaceutical compositions. A pharmaceutical composition disclosed herein can be administered to an individual in a single formulation or in separate formulations, for combined, simultaneous, or sequential administration.

Aspects of the present specification disclose a method of producing an immune response an individual. A method of producing an immune response disclosed herein comprises the step of administering a pharmaceutical composition disclosed herein. In aspects of this embodiment, a pharmaceutical composition comprises one or more components disclosed herein and/or scaffolds disclosed herein and/or self-assembled scaffolds disclosed herein and/or component-coated scaffolds disclosed herein and/or component-coated target particles disclosed herein. In other aspect of this embodiment, a pharmaceutical composition comprises one or more component-coated microorganism, such as, e.g., a proanthocyanidin-coated microorganism, such as, e.g., a proanthocyanidin-coated virus, a proanthocyanidin-coated toxoid, a proanthocyanidin-coated bacterium, a proanthocyanidin-coated mold, a proanthocyanidin-coated fungus, and/or a proanthocyanidin-coated protozoan. A pharmaceutical composition disclosed herein can be employed, without limitation, to generate an immune response in an organism which might otherwise have been killed by a vaccination with a toxin. In an aspect of this embodiment, a pharmaceutical composition disclosed herein is a vaccine composition. With the new and novel toxoids disclosed herein, the full set of antibodies comprising the immune response to a particular toxoid can be harvested and characterized, and one or more of them developed individually as potential therapeutics.

The production of an immune response by a pharmaceutical composition disclosed herein is generated through a composition-coated virus-like particle, including, without limitation, a protein cage, vault, a venom, a poison, an oligonucleotide or oligonucleotide analog, or other such synthetic or biological structure, and is used in place of a microorganism to generate an immune response. In a further embodiment a composition-coated virus-like particle is a toxoid.

A wide variety of viruses can be inhibited, including both DNA and RNA viruses of groups I through VII, enveloped and non-enveloped, such as but not limited to members of the genuses Adenoviridae, Arenaviridae, Bunyaviridae, Caliciviridae, Coronaviridae, Filoviridae, Flaviviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, and Togaviridae for animals, as well as corresponding viruses which infect plants, fungi, and other microorganisms as well as bacteriophages and related species.

In an embodiment, a pharmaceutical composition disclosed herein is administered to an individual along with an immunopotentiator. In an embodiment, an immunopotentiator is, without limitation, can be a cytokine (e.g. an interleukin such as IL-2 or IL-12), tumor necrosis factor (TNF-α), a male or female sex hormone, prolactin, a growth hormone, vitamin D, deoxycholic acid, a macrokine, imiquimod, resiquimod, and others.

A pharmaceutical composition disclosed herein may comprise a component disclosed herein and/or a scaffold disclosed herein and/or a self-assembled scaffold disclosed herein and/or a component-coated scaffold disclosed herein and/or a component-coated target particle disclosed herein in a therapeutically-effective amount. As used herein, the term “effective amount” is synonymous with “therapeutically-effective amount,” “effective dose,” or “therapeutically effective dose” and when used in reference to reducing the severity of a disease, a cancer, an infection, a skin ailment or other syndrome of an individual suffering from a disease, cancer, an infection, a skin ailment or other syndrome refers to the minimum dose of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein necessary to achieve the desired therapeutic effect and includes a dose sufficient to reduce the severity of a disease, a cancer, an infection, a skin ailment or other syndrome of an individual suffering from a disease, cancer, an infection, a skin ailment or other syndrome. The effectiveness of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein capable of reducing the severity of a disease, cancer, an infection, a skin ailment or other syndrome of an individual suffering from a disease, cancer, an infection, a skin ailment or other syndrome in an individual can be determined by observing an improvement in an individual based upon one or more clinical symptoms, and/or physiological indicators associated with reducing or maintaining the severity of a disease, cancer, an infection, a skin ailment or other syndrome of an individual suffering from a disease, a cancer, an infection, a skin ailment or other syndrome. Maintenance or a reduction of the severity of a disease, cancer, an infection, a skin ailment or other syndrome of an individual suffering from a disease, a cancer, an infection, a skin ailment or other syndrome can be indicated by a reduced need for a concurrent therapy. The effectiveness of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein capable of reducing or maintaining the severity of a disease, a cancer, an infection, a skin ailment or other syndrome of an individual suffering from a disease, a cancer, an infection, a skin ailment or other syndrome in an individual can be determined by observing an improvement in an individual based upon one or more clinical symptoms, and/or physiological indicators associated with a reduction or maintenance of the severity of a disease, cancer, an infection, a skin ailment or other syndrome of an individual suffering from a disease, cancer, an infection, a skin ailment or other syndrome.

In aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein reduces severity of a disease, cancer, an infection, a skin ailment or other syndrome of in an individual suffering from a disease, cancer, an infection, a skin ailment or other syndrome.

In aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein reduces severity of a disease, cancer, an infection, a skin ailment or other syndrome of in an individual suffering from a disease, cancer, an infection, a skin ailment or other syndrome by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein reduces severity of a disease, cancer, an infection, a skin ailment or other syndrome of in an individual suffering from a disease, cancer, an infection, a skin ailment or other syndrome by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a therapeutic compound disclosed herein severity of a disease, cancer, an infection, a skin ailment or other syndrome of in an individual suffering from a disease, cancer, an infection, a skin ailment or other syndrome by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be in the range of about 0.001 mg/kg/day to about 100 mg/kg/day. In aspects of this embodiment, an effective amount of a therapeutic compound disclosed herein may be, e.g., at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day. In other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be in the range of, e.g., about 0.001 mg/kg/day to about 10 mg/kg/day, about 0.001 mg/kg/day to about 15 mg/kg/day, about 0.001 mg/kg/day to about 20 mg/kg/day, about 0.001 mg/kg/day to about 25 mg/kg/day, about 0.001 mg/kg/day to about 30 mg/kg/day, about 0.001 mg/kg/day to about 35 mg/kg/day, about 0.001 mg/kg/day to about 40 mg/kg/day, about 0.001 mg/kg/day to about 45 mg/kg/day, about 0.001 mg/kg/day to about 50 mg/kg/day, about 0.001 mg/kg/day to about 75 mg/kg/day, or about 0.001 mg/kg/day to about 100 mg/kg/day.

In yet other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be in the range of, e.g., about 0.01 mg/kg/day to about 10 mg/kg/day, about 0.01 mg/kg/day to about 15 mg/kg/day, about 0.01 mg/kg/day to about 20 mg/kg/day, about 0.01 mg/kg/day to about 25 mg/kg/day, about 0.01 mg/kg/day to about 30 mg/kg/day, about 0.01 mg/kg/day to about 35 mg/kg/day, about 0.01 mg/kg/day to about 40 mg/kg/day, about 0.01 mg/kg/day to about 45 mg/kg/day, about 0.01 mg/kg/day to about 50 mg/kg/day, about 0.01 mg/kg/day to about 75 mg/kg/day, or about 0.01 mg/kg/day to about 100 mg/kg/day.

In still other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be in the range of, e.g., about 0.1 mg/kg/day to about 10 mg/kg/day, about 0.1 mg/kg/day to about 15 mg/kg/day, about 0.1 mg/kg/day to about 20 mg/kg/day, about 0.1 mg/kg/day to about 25 mg/kg/day, about 0.1 mg/kg/day to about 30 mg/kg/day, about 0.1 mg/kg/day to about 35 mg/kg/day, about 0.1 mg/kg/day to about 40 mg/kg/day, about 0.1 mg/kg/day to about 45 mg/kg/day, about 0.1 mg/kg/day to about 50 mg/kg/day, about 0.1 mg/kg/day to about 75 mg/kg/day, or about 0.1 mg/kg/day to about 100 mg/kg/day.

In other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be in the range of, e.g., about 1 mg/kg/day to about 10 mg/kg/day, about 1 mg/kg/day to about 15 mg/kg/day, about 1 mg/kg/day to about 20 mg/kg/day, about 1 mg/kg/day to about 25 mg/kg/day, about 1 mg/kg/day to about 30 mg/kg/day, about 1 mg/kg/day to about 35 mg/kg/day, about 1 mg/kg/day to about 40 mg/kg/day, about 1 mg/kg/day to about 45 mg/kg/day, about 1 mg/kg/day to about 50 mg/kg/day, about 1 mg/kg/day to about 75 mg/kg/day, or about 1 mg/kg/day to about 100 mg/kg/day.

In yet other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be in the range of, e.g., about 5 mg/kg/day to about 10 mg/kg/day, about 5 mg/kg/day to about 15 mg/kg/day, about 5 mg/kg/day to about 20 mg/kg/day, about 5 mg/kg/day to about 25 mg/kg/day, about 5 mg/kg/day to about 30 mg/kg/day, about 5 mg/kg/day to about 35 mg/kg/day, about 5 mg/kg/day to about 40 mg/kg/day, about 5 mg/kg/day to about 45 mg/kg/day, about 5 mg/kg/day to about 50 mg/kg/day, about 5 mg/kg/day to about 75 mg/kg/day, or about 5 mg/kg/day to about 100 mg/kg/day.

In other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be in the range of about 1 mg/day to about 3,000 mg/day. In aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be, e.g., at least 0.00001 mg/day, at least 0.00005 mg/day, at least 0.0001 mg/day, at least 0.0005 mg/day, 0.001 mg/day, at least 0.005 mg/day, at least 0.01 mg/day, at least 0.05 mg/day, at least 0.1 mg/day, at least 0.5 mg/day, at least at least at least 1 mg/day, at least 2 mg/day, at least 3 mg/day, at least 4 mg/day, at least 5 mg/day, at least 6 mg/day, at least 7 mg/day, at least 8 mg/day, at least 9 mg/day, at least 10 mg/day, at least 11 mg/day, at least 12 mg/day, at least 13 mg/day, at least 14 mg/day, at least 15 mg/day, at least 16 mg/day, at least 17 mg/day, at least 18 mg/day, at least 19 mg/day, at least 20 mg/day, at least 21 mg/day, at least 22 mg/day, at least 23 mg/day, at least 24 mg/day, at least 25 mg/day, at least 26 mg/day, at least 27 mg/day, at least 28 mg/day, at least 29 mg/day, at least 30 mg/day, at least 35 mg/day, at least 40 mg/day, at least 45 mg/day, at least 50 mg/day, at least 60 mg/day, at least 70 mg/day, at least 80 mg/day, at least 90 mg/day, at least 100 mg/day, at least 110 mg/day, at least 120 mg/day, at least 130 mg/day, at least 140 mg/day, at least 150 mg/day, at least 160 mg/day, at least 170 mg/day, at least 180 mg/day, at least 190 mg/day, at least 200 mg/day, at least 225 mg/day, at least 250 mg/day, at least 275 mg/day, at least 300 mg/day, at least 350 mg/day, at least 400 mg/day, at least 450 mg/day, at least 500 mg/day, at least 550 mg/day, at least 600 mg/day, at least 650 mg/day, at least 700 mg/day, at least 750 mg/day, at least 800 mg/day, at least 850 mg/day, at least 900 mg/day, at least 950 mg/day, at least 1,000 mg/day, at least 1,050 mg/day, at least 1,100 mg/day, at least 1,150 mg/day, at least 1,200 mg/day, at least 1,250 mg/day, at least 1,300 mg/day, at least 1,350 mg/day, at least 1,400 mg/day, at least 1,450 mg/day, at least 1,500 mg/day, at least 1,600 mg/day, at least 1,700 mg/day, at least 1,800 mg/day, at least 1,900 mg/day, at least 2,000 mg/day, at least 2,100 mg/day, at least 2,200 mg/day, at least 2,300 mg/day, at least 2,400 mg/day, at least 2,500 mg/day, at least 2,600 mg/day, at least 2,700 mg/day, at least 2,800 mg/day, at least 2,900 mg/day, or at least 3,000 mg/day.

In yet other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be between, e.g., about 0.0001 mg/day to about 225 mg/day, 1 mg/day to about 1,000 mg/day, about 5 mg/day to about 1,000 mg/day, about 10 mg/day to about 1,000 mg/day, about 15 mg/day to about 1,000 mg/day, about 20 mg/day to about 1,000 mg/day, about 25 mg/day to about 1,000 mg/day, about 30 mg/day to about 1,000 mg/day, about 40 mg/day to about 1,000 mg/day, about 50 mg/day to about 1,000 mg/day, about 100 mg/day to about 1,000 mg/day, about 150 mg/day to about 1,000 mg/day, about 200 mg/day to about 1,000 mg/day, about 250 mg/day to about 1,000 mg/day, about 300 mg/day to about 1,000 mg/day, about 350 mg/day to about 1,000 mg/day, about 400 mg/day to about 1,000 mg/day, about 450 mg/day to about 1,000 mg/day, about 500 mg/day to about 1,000 mg/day, about 50 mg/day to about 1,500 mg/day, about 100 mg/day to about 1,500 mg/day, about 150 mg/day to about 1,500 mg/day, about 200 mg/day to about 1,500 mg/day, about 250 mg/day to about 1,500 mg/day, about 300 mg/day to about 1,500 mg/day, about 350 mg/day to about 1,500 mg/day, about 400 mg/day to about 1,500 mg/day, about 450 mg/day to about 1,500 mg/day, about 500 mg/day to about 1,500 mg/day, about 1,000 mg/day to about 3,000 mg/day, about 1,100 mg/day to about 3,000 mg/day, about 1,200 mg/day to about 3,000 mg/day, about 1,3000 mg/day to about 3,000 mg/day, about 1,400 mg/day to about 3,000 mg/day, about 1,500 mg/day to about 3,000 mg/day, about 1,600 mg/day to about 3,000 mg/day, about 1,700 mg/day to about 3,000 mg/day, about 1,800 mg/day to about 3,000 mg/day, about 1,900 mg/day to about 3,000 mg/day, or about 2,000 mg/day to about 3,000 mg/day.

In other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be administered in the range of about 0.001 mg/kg/day to about 100 mg/kg/day. In aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be administered at a dose of, e.g., at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day.

In other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be administered at a dose of, e.g., about 0.001 mg/kg/day to about 10 mg/kg/day, about 0.001 mg/kg/day to about 15 mg/kg/day, about 0.001 mg/kg/day to about 20 mg/kg/day, about 0.001 mg/kg/day to about 25 mg/kg/day, about 0.001 mg/kg/day to about 30 mg/kg/day, about 0.001 mg/kg/day to about 35 mg/kg/day, about 0.001 mg/kg/day to about 40 mg/kg/day, about 0.001 mg/kg/day to about 45 mg/kg/day, about 0.001 mg/kg/day to about 50 mg/kg/day, about 0.001 mg/kg/day to about 75 mg/kg/day, or about 0.001 mg/kg/day to about 100 mg/kg/day.

In yet other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be in the range of, e.g., about 0.01 mg/kg/day to about 10 mg/kg/day, about 0.01 mg/kg/day to about 15 mg/kg/day, about 0.01 mg/kg/day to about 20 mg/kg/day, about 0.01 mg/kg/day to about 25 mg/kg/day, about 0.01 mg/kg/day to about 30 mg/kg/day, about 0.01 mg/kg/day to about 35 mg/kg/day, about 0.01 mg/kg/day to about 40 mg/kg/day, about 0.01 mg/kg/day to about 45 mg/kg/day, about 0.01 mg/kg/day to about 50 mg/kg/day, about 0.01 mg/kg/day to about 75 mg/kg/day, or about 0.01 mg/kg/day to about 100 mg/kg/day.

In still other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be in the range of, e.g., about 0.1 mg/kg/day to about 10 mg/kg/day, about 0.1 mg/kg/day to about 15 mg/kg/day, about 0.1 mg/kg/day to about 20 mg/kg/day, about 0.1 mg/kg/day to about 25 mg/kg/day, about 0.1 mg/kg/day to about 30 mg/kg/day, about 0.1 mg/kg/day to about 35 mg/kg/day, about 0.1 mg/kg/day to about 40 mg/kg/day, about 0.1 mg/kg/day to about 45 mg/kg/day, about 0.1 mg/kg/day to about 50 mg/kg/day, about 0.1 mg/kg/day to about 75 mg/kg/day, or about 0.1 mg/kg/day to about 100 mg/kg/day.

In other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be in the range of, e.g., about 1 mg/kg/day to about 10 mg/kg/day, about 1 mg/kg/day to about 15 mg/kg/day, about 1 mg/kg/day to about 20 mg/kg/day, about 1 mg/kg/day to about 25 mg/kg/day, about 1 mg/kg/day to about 30 mg/kg/day, about 1 mg/kg/day to about 35 mg/kg/day, about 1 mg/kg/day to about 40 mg/kg/day, about 1 mg/kg/day to about 45 mg/kg/day, about 1 mg/kg/day to about 50 mg/kg/day, about 1 mg/kg/day to about 75 mg/kg/day, or about 1 mg/kg/day to about 100 mg/kg/day.

In yet other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be in the range of, e.g., about 5 mg/kg/day to about 10 mg/kg/day, about 5 mg/kg/day to about 15 mg/kg/day, about 5 mg/kg/day to about 20 mg/kg/day, about 5 mg/kg/day to about 25 mg/kg/day, about 5 mg/kg/day to about 30 mg/kg/day, about 5 mg/kg/day to about 35 mg/kg/day, about 5 mg/kg/day to about 40 mg/kg/day, about 5 mg/kg/day to about 45 mg/kg/day, about 5 mg/kg/day to about 50 mg/kg/day, about 5 mg/kg/day to about 75 mg/kg/day, or about 5 mg/kg/day to about 100 mg/kg/day.

In other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be administered to an individual in the range of about 1 mg/day to about 3,000 mg/day. In aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be, e.g., at least 50 mg/day, at least 100 mg/day, at least 150 mg/day, at least 200 mg/day, at least 250 mg/day, at least 300 mg/day, at least 350 mg/day, at least 400 mg/day, at least 450 mg/day, at least 500 mg/day, at least 550 mg/day, at least 600 mg/day, at least 650 mg/day, at least 700 mg/day, at least 750 mg/day, at least 800 mg/day, at least 850 mg/day, at least 900 mg/day, at least 950 mg/day, at least 1,000 mg/day, at least 1.50 mg/day, at least 1,100 mg/day, at least 1,150 mg/day, at least 1,200 mg/day, at least 1,250 mg/day, at least 1,300 mg/day, at least 1,350 mg/day, at least 1,400 mg/day, at least 1,450 mg/day, at least 1,500 mg/day, at least 1,600 mg/day, at least 1,700 mg/day, at least 1,800 mg/day, at least 1,900 mg/day, at least 2,000 mg/day, at least 2,100 mg/day, at least 2,200 mg/day, at least 2,300 mg/day, at least 2,400 mg/day, at least 2,500 mg/day, at least 2,600 mg/day, at least 2,700 mg/day, at least 2,800 mg/day, at least 2,900 mg/day, or at least 3,000 mg/day.

In yet other aspects of this embodiment, a therapeutically effective amount of a component disclosed herein and/or a scaffold disclosed herein and/or a self-assembled scaffold disclosed herein and/or a component-coated scaffold disclosed herein and/or a component-coated target particle disclosed herein and/or a pharmaceutical composition disclosed herein may be between, e.g., about 50 mg/day to about 1,000 mg/day, about 100 mg/day to about 1,000 mg/day, about 150 mg/day to about 1,000 mg/day, about 200 mg/day to about 1,000 mg/day, about 250 mg/day to about 1,000 mg/day, about 300 mg/day to about 1,000 mg/day, about 350 mg/day to about 1,000 mg/day, about 400 mg/day to about 1,000 mg/day, about 450 mg/day to about 1,000 mg/day, about 500 mg/day to about 1,000 mg/day, about 50 mg/day to about 1,500 mg/day, about 100 mg/day to about 1,500 mg/day, about 150 mg/day to about 1,500 mg/day, about 200 mg/day to about 1,500 mg/day, about 250 mg/day to about 1,500 mg/day, about 300 mg/day to about 1,500 mg/day, about 350 mg/day to about 1,500 mg/day, about 400 mg/day to about 1,500 mg/day, about 450 mg/day to about 1,500 mg/day, about 500 mg/day to about 1,500 mg/day, about 1,000 mg/day to about 3,000 mg/day, about 1,100 mg/day to about 3,000 mg/day, about 1,200 mg/day to about 3,000 mg/day, about 1,3000 mg/day to about 3,000 mg/day, about 1,400 mg/day to about 3,000 mg/day, about 1,500 mg/day to about 3,000 mg/day, about 1,600 mg/day to about 3,000 mg/day, about 1,700 mg/day to about 3,000 mg/day, about 1,800 mg/day to about 3,000 mg/day, about 1,900 mg/day to about 3,000 mg/day, or about 2,000 mg/day to about 3,000 mg/day.

Dosing can be single dosage or cumulative (serial dosing), and can be readily determined by one skilled in the art. For instance, reducing or maintaining the severity of a disease, cancer, an infection, a skin ailment or other syndrome of an individual suffering from a disease, cancer, an infection, a skin ailment or other syndrome in an individual may comprise a one-time administration of an effective dose of a pharmaceutical composition disclosed herein. Alternatively, reducing or maintaining the severity of a disease, cancer, an infection, a skin ailment or other syndrome of an individual suffering from a disease, cancer, an infection, a skin ailment or other syndrome in an individual may comprise multiple administrations of an effective dose of a pharmaceutical composition carried out over a range of time periods, such as, e.g., once daily, twice daily, thrice daily, once every few days, or once weekly. The timing of administration can vary from individual to individual, depending upon such factors as the severity of an individual's symptoms. For example, an effective dose of a pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

A pharmaceutical composition produced using the methods disclosed herein may be a liquid formulation, semi-solid formulation, or a solid formulation. A formulation disclosed herein can be produced in a manner to form one phase, such as, e.g., an oil or a solid. Alternatively, a formulation disclosed herein can be produced in a manner to form two phase, such as, e.g., an emulsion. A pharmaceutical composition disclosed herein intended for such administration may be prepared according to any method known to the art for the manufacture of pharmaceutical compositions. Semi-solid formulations suitable for topical administration include, without limitation, ointments, creams, salves, and gels.

Aspects of the present specification may also be described as follows:
1. A composition comprising a scaffold and a coating which is capable of transferring the coating to an exposed surface.
2. The composition according to embodiment 1, wherein the coating component which is capable of coating exposed surfaces is obtained as an extract from a fruit.
3. The composition according to embodiment 1 or embodiment 2, wherein the fruit is a grape or a persimmon.
4. The composition according to any one of embodiments 1-3, wherein the component in an extract that is capable of coating exposed surfaces is a tannin.
5. The composition of claim 4, wherein the tannin is naturally occurring or synthetically manufactured.
6. The composition according to embodiment 4, wherein the tannin is obtained or extracted from Aceraceae, Actinidiaceae, Anacardiaceae, Bixaceae, Burseraceae, Combretaceae, Dipterocarpaceae, Ericaceae, Grossulariaceae, Myricaceae, Najadaceae, Vitaceae, Typhaceae, or any combination thereof.
7. The composition according to embodiment 4, wherein the tannin is a pseudotannin.
8. The composition according to embodiment 4, wherein the tannin is a proanthocyanidin.
9. The composition according to embodiment 8, wherein the proanthocyanidin is comprised of polymers of 2 to 100 (or more) flavan-3-ol units joined by a bond that are not susceptible to being cleaved by hydrolysis.
10. The composition according to any one of embodiments 4-10, wherein the tannin has a molecular weight of at least 100 Daltons, at least 200 Daltons, at least 300 Daltons, at least 400 Daltons, at least 500 Daltons, at least 600 Daltons, at least 700 Daltons, at least 800 Daltons, at least 900 Daltons, at least 1000 Daltons, at least 1250 Daltons, at least 1500 Daltons, at least 1750 Daltons, at least 2000 Daltons, at least 2250 Daltons, at least 2500 Daltons, at least 2750 Daltons, at least 3000 Daltons, at least 3250 Daltons, at least 3500 Daltons, at least 3750 Daltons, at least 4000 Daltons, at least 4250 Daltons, at least 4500 Daltons, at least 4750 Daltons, at least 5000 Daltons, at least 5250 Daltons, at least 5500 Daltons, at least 5750 Daltons, 6000 Daltons, 6250 Daltons, 6500 Daltons, at least 6750 Daltons, at least 7000 Daltons, at least 7250 Daltons, at least 7500 Daltons, at least 7750 Daltons, at least 8000 Daltons, at least 8250 Daltons, at least 8500 Daltons, at least 8750 Daltons, at least 9000 Daltons, at least 9250 Daltons, at least 9500 Daltons, at least 9750 Daltons, at least 10000 Daltons, at least 10250 Daltons, at least 10500 Daltons, at least 10750 Daltons, at least 11000 Daltons, at least 11250 Daltons, at least 11500 Daltons, at least 11750 Daltons, at least 12000 Daltons, at least 12250 Daltons, at least 12500 Daltons, at least 12750 Daltons, at least 13000 Daltons, at least 13250 Daltons, at least 13500 Daltons, at least 13750 Daltons, at least 14000 Daltons, at least 14250 Daltons, at least 14500 Daltons, at least 14750 Daltons, at least 15000 Daltons, at least 15250 Daltons, at least 15500 Daltons, at least 15750 Daltons, at least 16000 Daltons, at least 16250 Daltons, at least 16500 Daltons, at least 16750 Daltons, at least 17000 Daltons, at least 17250 Daltons, at least 17500 Daltons, at least 17750 Daltons, at least 18000 Daltons, at least 18250 Daltons, at least 18500 Daltons, at least 18750 Daltons, at least 19000 Daltons, at least 19250 Daltons, at least 19500 Daltons, at least 19750 Daltons, at least 20000 Daltons, at least 20250 Daltons, at least 20500 Daltons, at least 20750 Daltons, at least 21000 Daltons, at least 22000 Daltons, at least 23000 Daltons, at least 24000 Daltons, at least 25000 Daltons, or more.
11. The composition according to any one of embodiments 1-10, wherein the composition attenuates the pathogenicity of the object in a biologic organism to which the object is administered to allow for the propagation of an immune response; attenuates the toxic effect of a substance or biologic organism; provide a lubricant effect to the coated object; protects the coated object from degradation such as wear, abrasion, oxidation, or other processes; increases or decreases the electrical conductivity or resistance of an object; produces images, patterns, graphics or text on the surfaces of objects; increases or decreases the disconnection or desorption of species from objects' surfaces; increases or decreases the biological activity or action of living organisms; and/or exposes or masks the surfaces of objects from incident radiation or chemical or mechanical treatment.
12. The composition according to any one of embodiments 1-11, wherein the scaffold is spherical, cubic, rectangular, cylindrical, helical, triangular, pyrimidal, and/or tetrahedral in shape.
13. The composition according to any one of embodiments 1-12, wherein the scaffold is comprised of a biological organism, a protein, a fat, a carbohydrate, a metal object, a composite object, a glass bead, a fiber, a bead, a peptide, a nucleic acid, a peptide nucleic acid, a virus particle, a lipid, a micelle, a polymer latex, a liposome, and/or a rubber object.
14. The composition according to any one of embodiments 1-13, wherein the bead is a Sephadex bead, a dextran bead, a poly(styrene) bead, a silica bead, and/or a diethylaminoethylcellulose bead.
15. The composition according to any one of embodiments 1-14, wherein the scaffold has a diameter of at least 1 nm (nanometer), at least 2 nm, at least 3 nm, at least 4 nm, at least 5 nm, at least 10 nm, at least 15 nm, at least 20 nm, at least 25 nm, at least 30 nm, at least 35 nm, at least 40 nm, at least 45 nm, at least 50 nm, at least 55 nm, at least 60 nm, at least 65 nm, at least 70 nm, at least 75 nm, at least 80 nm, at least 85 nm, at least 90 nm, at least 95 nm, at least 100 nm, at least 150 nm, at least 200 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1.0 μm (micrometers), at least 2 μm, at least 3 μm, at least 4 μm, at least 5 μm, at least 6 μm, at least 7 μm, at least 8 μm, at least 9 μm, at least 10 μm, at least 11 μm, at least 12 μm, at least 13 μm, at least 14 μm, at least 15 μm, at least 16 μm, at least 17 μm, at least 18 μm, at least 19 μm, at least 20 μm, at least 21 μm, at least 22 μm, at least 23 μm, at least 24 μm, at least 25 μm, at least 26 μm, at least 27 μm, at least 28 μm, at least 29 μm, at least 30 μm, at least 31 μm, at least 32 μm, at least 33 μm, at least 34 μm, at least 35 μm, at least 36 μm, at least 37 μm, at least 38 μm, at least 39 μm, at least 40 μm, at least 41 μm, at least 42 μm, at least 43 μm, at least 44 μm, at least 45 μm, at least 46 μm, at least 47 μm, at least 48 μm, at least 49 μm, at least 50 μm, at least 51 μm, at least 52 μm, at least 53 μm, at least 54 μm, at least 55 μm, at least 56 μm, at least 57 μm, at least 58 μm, at least 59 μm, at least 60 μm, at least 61 μm, at least 62 μm, at least 63 μm, at least 64 μm, at least 65 μm, at least 66 μm, at least 67 μm, at least 68 μm, at least 69 μm, at least 70 μm, at least 71 μm, at least 72 μm, at least 73 μm, at least 74 μm, at least 75 μm, at least 76 μm, at least 77 μm, at least 78 μm, at least 79 μm, at least 80 μm, at least 81 μm, at least 82 μm, at least 83 μm, at least 84 μm, at least 85 μm, at least 86 μm, at least 87 μm, at least 88 μm, at least 89 μm, at least 90 μm, at least 91 μm, at least 92 μm, at least 93 μm, at least 94 μm, at least 95 μm, at least 96 μm, at least 97 μm, at least 98 μm, at least 99 μm, at least 100 μm, or more.
16. The composition according to any one of embodiments 1-15, wherein the composition is administered to an individual.
17. The composition according to embodiment 16, wherein the individual is an animal is a mammal, a reptile, a bird, a marsupial or other animal.
18. The composition according to embodiment 17, wherein the mammal is a human, a cat, a dog, a cow, a horse, a goat, a sheep, a pig or other domestic or non-domesticated animal.
19. The composition according to any one of embodiments 1-18, wherein the scaffold is comprised of a proanthocyanidin.
20. The composition according to any one of embodiments 1-19, wherein the scaffold is a particle or particulate lattice.
21. The composition according to any one of embodiments 1-20, wherein the composition is capable of transferring an extract that is capable of coating exposed surfaces from the scaffold to another object.
22. The composition according to embodiment 21, wherein the transfer occurs as a result of dialysis, precipitation, or electroprecipitation.
23. The composition according to embodiment 21, wherein the transfer of the coating from the scaffold to the exposed surface occurs as a result of diafiltration or tangential-flow filtration.
24. The composition according to embodiment 21, wherein the transfer of the coating from the scaffold to the exposed surface occurs as a result of spray-drying or supercritical fluid evaporation.
25. The composition according to embodiment 21, wherein the transfer of an extract that is capable of coating exposed surfaces is to a microorganism, a protein cage, an antibody, a vault, a venom, or a poison.
26. The composition according to embodiment 25, wherein the microorganism is a bacterium, a virus, a mold, a fungus, a sub-cellular organelle, or a bacteriophage.
27. The composition according to any one of embodiments 1-26, wherein the extract that is capable of coating exposed surfaces is from a grape seed extract.
28. The composition according to embodiment 24, wherein the coated microorganism is a toxoid.
29. The composition according to any one of embodiments 1-28, wherein the composition is a therapeutic compound.
30. The composition according to embodiment 29, wherein the therapeutic compound is capable of reducing the severity of a disease, cancer, an infection, a skin ailment or other syndrome.
31. The composition according to embodiment 29 or embodiment 30, wherein the therapeutic compound is capable of reducing the severity of a disease, cancer, infection, skin ailment or other syndrome of an individual suffering from a disease, cancer, infection, skin ailment or other syndrome by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a therapeutic compound capable of reducing the severity of a disease, cancer, infection, skin ailment or other syndrome in an individual suffering from a disease, cancer, infection, skin ailment or other syndrome by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.
32. The composition according to any one of embodiments 29-31, wherein the therapeutic compound and one or more pharmacologically-acceptable carriers comprise a pharmaceutical composition.
33. The composition according to embodiment 32, wherein a pharmacologically-acceptable carrier comprises a pharmacologically acceptable vehicle, stabilizer, diluent, additive, auxiliary, or excipient.
34. The composition according to any one of embodiments 29-33, wherein a pharmaceutical composition is administered to a patient for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.
35. The composition according to embodiment 32, wherein an individual administered a pharmaceutical composition has the administration stopped for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more followed by administration of the pharmaceutical composition to the individual.
36. The composition according to any one of embodiments 1-35, wherein the composition maintains or reduces the severity of a disease caused by Adenoviridae, Arenaviridae, Bunyaviridae, Caliciviridae, Coronaviridae, Filoviridae, Flaviviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, and Togaviridae.
37. The composition according to any one of embodiments 1-36, wherein the composition comprises a proanthocyanidin-coated virus.
38. The composition according to any one of embodiments 1-37, wherein the scaffold and an extract that is capable of coating exposed surfaces are incubated for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, at least 20 minutes, at least 21 minutes, at least 22 minutes, at least 23 minutes, at least 24 minutes, at least 25 minutes, at least 26 minutes, at least 27 minutes, at least 28 minutes, at least 29 minutes, at least 30 minutes, at least 31 minutes, at least 32 minutes, at least 33 minutes, at least 34 minutes, at least 35 minutes, at least 36 minutes, at least 37 minutes, at least 38 minutes, at least 39 minutes, at least 40 minutes, at least 41 minutes, at least 42 minutes, at least 43 minutes, at least 44 minutes, at least 45 minutes, at least 46 minutes, at least 47 minutes, at least 48 minutes, at least 49 minutes, at least 50 minutes, at least 51 minutes, at least 52 minutes, at least 53 minutes, at least 54 minutes, at least 55 minutes, at least 56 minutes, at least 57 minutes, at least 58 minutes, at least 59 minutes, at least 60 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 37 hours, at least 38 hours, at least 39 hours, at least 40 hours, at least 41 hours, at least 42 hours, at least 43 hours, at least 44 hours, at least 45 hours, at least 46 hours, at least 47 hours, at least 48 hours, or more.
39. The composition according to any one of embodiments 1-38, wherein the pharmaceutical composition is administered to an individual orally, sublingually, enterally, subcutaneously, intramuscularly, rectally, transdermally, intravaginally, intranasally, through eye drops, through ear drops, through intravenous injection, through insufflation, through depot, or by topical application.
40. The composition according to any one of embodiments 1-39, wherein a pharmaceutical composition includes an adjuvant.
41. The composition according to embodiment 40, wherein an adjuvant is added to a pharmaceutical composition in an amount of at least 10% (v/v), at least 20% (v/v), at least 30% (v/v), at least 35% (v/v), at least 40% (v/v), at least 45% (v/v), at least 50% (v/v), at least 55% (v/v), at least 60% (v/v), at least 65% (v/v), at least 70% (v/v), at least 75% (v/v), at least 80% (v/v), at least 85% (v/v), at least 90% (v/v), at least 95% (v/v), or at least 99% (v/v). In other aspects of this embodiment, a pharmaceutical composition disclosed herein may comprise an adjuvant in an amount in a range of, e.g., about 30% (v/v) to about 99% (v/v), about 35% (v/v) to about 99% (v/v), about 40% (v/v) to about 99% (v/v), about 45% (v/v) to about 99% (v/v), about 50% (v/v) to about 99% (v/v), about 30% (v/v) to about 98% (v/v), about 35% (v/v) to about 98% (v/v), about 40% (v/v) to about 98% (v/v), about 45% (v/v) to about 98% (v/v), about 50% (v/v) to about 98% (v/v), about 30% (v/v) to about 95% (v/v), about 35% (v/v) to about 95% (v/v), about 40% (v/v) to about 95% (v/v), about 45% (v/v) to about 95% (v/v), or about 50% (v/v) to about 95% (v/v).
42. The composition according to embodiment 40, wherein the adjuvant in an amount in a range of about 70% (v/v) to about 97% (v/v), about 75% (v/v) to about 97% (v/v), about 80% (v/v) to about 97% (v/v), about 85% (v/v) to about 97% (v/v), about 88% (v/v) to about 97% (v/v), about 89% (v/v) to about 97% (v/v), about 90% (v/v) to about 97% (v/v), about 75% (v/v) to about 96% (v/v), about 80% (v/v) to about 96% (v/v), about 85% (v/v) to about 96% (v/v), about 88% (v/v) to about 96% (v/v), about 89% (v/v) to about 96% (v/v), about 90% (v/v) to about 96% (v/v), about 75% (v/v) to about 93% (v/v), about 80% (v/v) to about 93% (v/v), about 85% (v/v) to about 93% (v/v), about 88% (v/v) to about 93% (v/v), about 89% (v/v) to about 93% (v/v), or about 90% (v/v) to about 93% (v/v).
43. The composition according to embodiment 40, wherein an adjuvant is keyhole limpet hemocyanin, an oil emulsion, a toxins, an aluminum salt, a lipid, or aluminum hydroxide.
44. The composition according to any one of embodiments 29-43, wherein the pharmaceutical composition comprises a sustained release therapeutic compound delivery platform.
45. The composition according to embodiment 44, wherein a sustained release therapeutic compound delivery platform releases a therapeutic compound with substantially zero-order release kinetics over a period of, e.g., about 7 days after administration, about 15 days after administration, about 30 days after administration, about 45 days after administration, about 60 days after administration, about 75 days after administration, or about 90 days after administration.
46. The composition according to embodiment 44, wherein a sustained release therapeutic compound delivery platform releases a therapeutic compound with substantially zero-order release kinetics over a period of, e.g., at least 7 days after administration, at least 15 days after administration, at least 30 days after administration, at least 45 days after administration, at least 60 days after administration, at least 75 days after administration, or at least 90 days after administration.
47. The composition according to embodiment 44, wherein a therapeutic compound delivery platform releases a therapeutic compound with substantially zero-order release kinetics over a period of about 1 day after administration, about 2 days after administration, about 3 days after administration, about 4 days after administration, about 5 days after administration, or about 6 days after administration.
48. The composition according to embodiment 44, wherein a therapeutic compound delivery platform releases a therapeutic compound with substantially zero-order release kinetics over a period of at most 1 day after administration, at most 2 days after administration, at most 3 days after administration, at most 4 days after administration, at most 5 days after administration, or at most 6 days after administration.
49. The composition according to any one of embodiments 29-48, wherein a therapeutically-effective amount of a therapeutic compound reduces the severity of a disease, cancer, an infection, a skin ailment or other syndrome of an individual suffering from a disease, cancer, infection, skin ailment or other syndrome by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.
50. The composition according to any one of embodiments 29-48, wherein a therapeutically-effective amount of a therapeutic compound reduces the severity of a disease, cancer, infection, skin ailment, or other syndrome of an individual suffering from a disease, cancer, infection, skin ailment or other syndrome by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, or at most 100%.
51. The composition according to any one of embodiments 29-48, wherein a therapeutically-effective amount of a therapeutic compound reduces the severity of a disease, cancer, infection, skin ailment, or other syndrome of an individual suffering from a disease, cancer, infection, skin ailment, or other syndrome by about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.
52. The composition according to any one of embodiments 29-51, wherein the pharmaceutical composition comprises one or more active ingredients.
53. The composition according to embodiment 52, wherein the one or more active ingredients are present at a concentration of at least about 0.1% (w/v), or alternatively at least about 0.01%, 0.02%, 0.05%, 0.075%, 0.2%, 0.3%, 0.4%, 0.5%, 0.6%, 0.7%, 0.8%, 0.9%, 1%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, 19%, 19.25%, 19.5%, 19.75%, 20%, 20.25%, 20.5%, 20.75%, 21%, 21.25%, 21.5%, 21.75%, 22%, 22.25%, 22.5%, 22.75%, 23%, 23.25%, 23.5%, 23.75%, 24%, 24.25%, 24.5%, 24.75%, 25%, 26%, 27%, 28%, 29%, 30%, 31%, 32%, 33%, 34%, 35%, 40%, or more (w/v) of the skin care composition.
54. The composition according to embodiment 52, wherein the one or more active ingredients are present at a concentration of about 0.1% (w/v) to about 40%, or alternatively at about 0.01% to about 25%, 0.02% to about 25%, 0.05% to about 25%, 0.075% to about 25%, 0.2% to about 25%, 0.3% to about 25%, 0.4% to about 25%, 0.5% to about 25%, 0.6% to about 25%, 0.7% to about 25%, 0.8% to about 25%, 0.9% to about 25%, 1% to about 25%, 1.5% to about 25%, 1.75% to about 25%, 2% to about 25%, 2.25% to about 25%, 2.5% to about 25%, 2.75% to about 25%, 3% to about 25%, 3.25% to about 25%, 3.5% to about 25%, 3.75% to about 25%, 4% to about 25%, 4.25% to about 25%, 4.5% to about 25%, 4.75% to about 25%, 5% to about 25%, 5.25% to about 25%, 5.5% to about 25%, 5.75% to about 25%, 6% to about 25%, 6.25% to about 25%, 6.5% to about 25%, 6.75% to about 25%, 7% to about 25%, 7.25% to about 25%, 7.5% to about 25%, 7.75% to about 25%, 8% to about 25%, 8.25% to about 25%, 8.5% to about 25%, 8.75% to about 25%, 9% to about 25%, 9.25% to about 25%, 9.5% to about 25%, 9.75% to about 25%, 10% to about 25%, 10.25% to about 25%, 10.5% to about 25%, 10.75% to about 25%, 11% to about 25%, 11.25% to about 25%, 11.5% to about 25%, 11.75% to about 25%, 12% to about 25%, 12.25% to about 25%, 12.5% to about 25%, 12.75% to about 25%, 13% to about 25%, 13.25% to about 25%, 13.5% to about 25%, 13.75% to about 25%, 14% to about 25%, 14.25% to about 25%, 14.5% to about 25%, 14.75% to about 25%, 15% to about 25%, 15.25% to about 25%, 15.5% to about 25%, 15.75% to about 25%, 16% to about 25%, 16.25% to about 25%, 16.5% to about 25%, 16.75% to about 25%, 17% to about 25%, 17.25% to about 25%, 17.5% to about 25%, 17.75% to about 25%, 18% to about 25%, 18.25% to about 25%, 18.5% to about 25%, 18.75% to about 25%, 19% to about 25%, 19.25% to about 25%, 19.5% to about 25%, 19.75% to about 25%, 20% to about 25%, 20.25% to about 25%, 20.5% to about 25%, 20.75% to about 25%, 5% to about 20%, 6% to about 20%, 7% to about 20%, 8% to about 20%, 9% to about 20%, 10% to about 20%, 11% to about 20%, 12% to about 20%, 13%, to about 20%, 14% to about 20%, 15% to about 20%, 5% to about 15%, 6% to about 15%, 7% to about 15%, 8% to about 15%, 9% to about 15%, 10% to about 15%, 11% to about 15%, 12% to about 15%, 13%, to about 15%, 14% to about 15% (w/v).
55. The composition according to any one of embodiments 29-54, wherein a therapeutically-effective amount of a therapeutic compound is in the range of about 0.001 mg/kg/day to about 100 mg/kg/day.
56. The composition according to any one of embodiments 29-54, wherein an effective amount of a therapeutic compound is at least 0.00001 mg/day, at least 0.001 mg/day, at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day.
57. The composition according to any one of embodiments 29-54, wherein an effective amount of a therapeutic compound is in the range of about 0.001 mg/kg/day to about 10 mg/kg/day, about 0.001 mg/kg/day to about 15 mg/kg/day, about 0.001 mg/kg/day to about 20 mg/kg/day, about 0.001 mg/kg/day to about 25 mg/kg/day, about 0.001 mg/kg/day to about 30 mg/kg/day, about 0.001 mg/kg/day to about 35 mg/kg/day, about 0.001 mg/kg/day to about 40 mg/kg/day, about 0.001 mg/kg/day to about 45 mg/kg/day, about 0.001 mg/kg/day to about 50 mg/kg/day, about 0.001 mg/kg/day to about 75 mg/kg/day, or about 0.001 mg/kg/day to about 100 mg/kg/day.
58. The composition according to any one of embodiments 29-54, wherein an effective amount of a therapeutic compound is in the range of about 0.01 mg/kg/day to about 10 mg/kg/day, about 0.01 mg/kg/day to about 15 mg/kg/day, about 0.01 mg/kg/day to about 20 mg/kg/day, about 0.01 mg/kg/day to about 25 mg/kg/day, about 0.01 mg/kg/day to about 30 mg/kg/day, about 0.01 mg/kg/day to about 35 mg/kg/day, about 0.01 mg/kg/day to about 40 mg/kg/day, about 0.01 mg/kg/day to about 45 mg/kg/day, about 0.01 mg/kg/day to about 50 mg/kg/day, about 0.01 mg/kg/day to about 75 mg/kg/day, or about 0.01 mg/kg/day to about 100 mg/kg/day.
59. The composition according to any one of embodiments 29-54, wherein, an effective amount of a therapeutic compound is in the range of about 0.1 mg/kg/day to about 10 mg/kg/day, about 0.1 mg/kg/day to about 15 mg/kg/day, about 0.1 mg/kg/day to about 20 mg/kg/day, about 0.1 mg/kg/day to about 25 mg/kg/day, about 0.1 mg/kg/day to about 30 mg/kg/day, about 0.1 mg/kg/day to about 35 mg/kg/day, about 0.1 mg/kg/day to about 40 mg/kg/day, about 0.1 mg/kg/day to about 45 mg/kg/day, about 0.1 mg/kg/day to about 50 mg/kg/day, about 0.1 mg/kg/day to about 75 mg/kg/day, or about 0.1 mg/kg/day to about 100 mg/kg/day.
60. The composition according to any one of embodiments 29-54, wherein a therapeutically effective amount of a therapeutic compound is in the range of about 1 mg/day to about 3,000 mg/day.
61. The composition according to any one of embodiments 29-54, wherein an effective amount of a therapeutic compound is at least 1 mg/day, at least 5 mg/day, at least 10 mg/day, at least 15 mg/day, at least 20 mg/day, at least 25 mg/day, at least 30 mg/day, at least 40 mg/day, at least 50 mg/day, at least 100 mg/day, at least 150 mg/day, at least 200 mg/day, at least 250 mg/day, at least 300 mg/day, at least 350 mg/day, at least 400 mg/day, at least 450 mg/day, at least 500 mg/day, at least 550 mg/day, at least 600 mg/day, at least 650 mg/day, at least 700 mg/day, at least 750 mg/day, at least 800 mg/day, at least 850 mg/day, at least 900 mg/day, at least 950 mg/day, at least 1,000 mg/day, at least 1.50 mg/day, at least 1,100 mg/day, at least 1,150 mg/day, at least 1,200 mg/day, at least 1,250 mg/day, at least 1,300 mg/day, at least 1,350 mg/day, at least 1,400 mg/day, at least 1,450 mg/day, at least 1,500 mg/day, at least 1,600 mg/day, at least 1,700 mg/day, at least 1,800 mg/day, at least 1,900 mg/day, at least 2,000 mg/day, at least 2,100 mg/day, at least 2,200 mg/day, at least 2,300 mg/day, at least 2,400 mg/day, at least 2,500 mg/day, at least 2,600 mg/day, at least 2,700 mg/day, at least 2,800 mg/day, at least 2,900 mg/day, or at least 3,000 mg/day.
62. The composition according to any one of embodiments 29-54, wherein an effective amount of a therapeutic compound disclosed herein may be between, e.g., about 1 mg/day to about 1,000 mg/day, about 5 mg/day to about 1,000 mg/day, about 10 mg/day to about 1,000 mg/day, about 15 mg/day to about 1,000 mg/day, about 20 mg/day to about 1,000 mg/day, about 25 mg/day to about 1,000 mg/day, about 30 mg/day to about 1,000 mg/day, about 40 mg/day to about 1,000 mg/day, about 50 mg/day to about 1,000 mg/day, about 100 mg/day to about 1,000 mg/day, about 150 mg/day to about 1,000 mg/day, about 200 mg/day to about 1,000 mg/day, about 250 mg/day to about 1,000 mg/day, about 300 mg/day to about 1,000 mg/day, about 350 mg/day to about 1,000 mg/day, about 400 mg/day to about 1,000 mg/day, about 450 mg/day to about 1,000 mg/day, about 500 mg/day to about 1,000 mg/day, about 50 mg/day to about 1,500 mg/day, about 100 mg/day to about 1,500 mg/day, about 150 mg/day to about 1,500 mg/day, about 200 mg/day to about 1,500 mg/day, about 250 mg/day to about 1,500 mg/day, about 300 mg/day to about 1,500 mg/day, about 350 mg/day to about 1,500 mg/day, about 400 mg/day to about 1,500 mg/day, about 450 mg/day to about 1,500 mg/day, about 500 mg/day to about 1,500 mg/day, about 1,000 mg/day to about 3,000 mg/day, about 1,100 mg/day to about 3,000 mg/day, about 1,200 mg/day to about 3,000 mg/day, about 1,3000 mg/day to about 3,000 mg/day, about 1,400 mg/day to about 3,000 mg/day, about 1,500 mg/day to about 3,000 mg/day, about 1,600 mg/day to about 3,000 mg/day, about 1,700 mg/day to about 3,000 mg/day, about 1,800 mg/day to about 3,000 mg/day, about 1,900 mg/day to about 3,000 mg/day, or about 2,000 mg/day to about 3,000 mg/day.
63. The composition according to any one of embodiments 29-54, wherein a therapeutically-effective amount of a component capable of coating an exposed surface is administered in the range of about 0.00001 mg/kg/day to about 100 mg/kg/day.
64. The composition according to any one of embodiments 29-54, wherein a component capable of coating an exposed surface is administered at a dose of at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day.
65. The composition according to any one of embodiments 29-54, wherein a component capable of coating an exposed surface may be administered at a dose of about 0.001 mg/kg/day to about 10 mg/kg/day, about 0.001 mg/kg/day to about 15 mg/kg/day, about 0.001 mg/kg/day to about 20 mg/kg/day, about 0.001 mg/kg/day to about 25 mg/kg/day, about 0.001 mg/kg/day to about 30 mg/kg/day, about 0.001 mg/kg/day to about 35 mg/kg/day, about 0.001 mg/kg/day to about 40 mg/kg/day, about 0.001 mg/kg/day to about 45 mg/kg/day, about 0.001 mg/kg/day to about 50 mg/kg/day, about 0.001 mg/kg/day to about 75 mg/kg/day, or about 0.001 mg/kg/day to about 100 mg/kg/day.
66. The composition according to any one of embodiments 29-54, wherein a therapeutic compound is in the range of about 0.01 mg/kg/day to about 10 mg/kg/day, about 0.01 mg/kg/day to about 15 mg/kg/day, about 0.01 mg/kg/day to about 20 mg/kg/day, about 0.01 mg/kg/day to about 25 mg/kg/day, about 0.01 mg/kg/day to about 30 mg/kg/day, about 0.01 mg/kg/day to about 35 mg/kg/day, about 0.01 mg/kg/day to about 40 mg/kg/day, about 0.01 mg/kg/day to about 45 mg/kg/day, about 0.01 mg/kg/day to about 50 mg/kg/day, about 0.01 mg/kg/day to about 75 mg/kg/day, about 0.01 mg/kg/day to about 100 mg/kg/day, about 5 mg/kg/day to about 10 mg/kg/day, about 5 mg/kg/day to about 15 mg/kg/day, about 5 mg/kg/day to about 20 mg/kg/day, about 5 mg/kg/day to about 25 mg/kg/day, about 5 mg/kg/day to about 30 mg/kg/day, about 5 mg/kg/day to about 35 mg/kg/day, about 5 mg/kg/day to about 40 mg/kg/day, about 5 mg/kg/day to about 45 mg/kg/day, about 5 mg/kg/day to about 50 mg/kg/day, about 5 mg/kg/day to about 75 mg/kg/day, or about 5 mg/kg/day to about 100 mg/kg/day.
67. The composition according to any one of embodiments 29-54, wherein a therapeutic compound is administered to an individual in the range of about 1 mg/day to about 3,000 mg/day.
68. The composition according to any one of embodiments 29-54, wherein a therapeutic compound is administered to an individual in a range of at least 50 mg/day, at least 100 mg/day, at least 150 mg/day, at least 200 mg/day, at least 250 mg/day, at least 300 mg/day, at least 350 mg/day, at least 400 mg/day, at least 450 mg/day, at least 500 mg/day, at least 550 mg/day, at least 600 mg/day, at least 650 mg/day, at least 700 mg/day, at least 750 mg/day, at least 800 mg/day, at least 850 mg/day, at least 900 mg/day, at least 950 mg/day, at least 1,000 mg/day, at least 1.50 mg/day, at least 1,100 mg/day, at least 1,150 mg/day, at least 1,200 mg/day, at least 1,250 mg/day, at least 1,300 mg/day, at least 1,350 mg/day, at least 1,400 mg/day, at least 1,450 mg/day, at least 1,500 mg/day, at least 1,600 mg/day, at least 1,700 mg/day, at least 1,800 mg/day, at least 1,900 mg/day, at least 2,000 mg/day, at least 2,100 mg/day, at least 2,200 mg/day, at least 2,300 mg/day, at least 2,400 mg/day, at least 2,500 mg/day, at least 2,600 mg/day, at least 2,700 mg/day, at least 2,800 mg/day, at least 2,900 mg/day, or at least 3,000 mg/day.
69. The composition according to any one of embodiments 29-54, wherein a therapeutic compound is administered to an individual in a range of about 50 mg/day to about 1,000 mg/day, about 100 mg/day to about 1,000 mg/day, about 150 mg/day to about 1,000 mg/day, about 200 mg/day to about 1,000 mg/day, about 250 mg/day to about 1,000 mg/day, about 300 mg/day to about 1,000 mg/day, about 350 mg/day to about 1,000 mg/day, about 400 mg/day to about 1,000 mg/day, about 450 mg/day to about 1,000 mg/day, about 500 mg/day to about 1,000 mg/day, about 50 mg/day to about 1,500 mg/day, about 100 mg/day to about 1,500 mg/day, about 150 mg/day to about 1,500 mg/day, about 200 mg/day to about 1,500 mg/day, about 250 mg/day to about 1,500 mg/day, about 300 mg/day to about 1,500 mg/day, about 350 mg/day to about 1,500 mg/day, about 400 mg/day to about 1,500 mg/day, about 450 mg/day to about 1,500 mg/day, about 500 mg/day to about 1,500 mg/day, about 1,000 mg/day to about 3,000 mg/day, about 1,100 mg/day to about 3,000 mg/day, about 1,200 mg/day to about 3,000 mg/day, about 1,3000 mg/day to about 3,000 mg/day, about 1,400 mg/day to about 3,000 mg/day, about 1,500 mg/day to about 3,000 mg/day, about 1,600 mg/day to about 3,000 mg/day, about 1,700 mg/day to about 3,000 mg/day, about 1,800 mg/day to about 3,000 mg/day, about 1,900 mg/day to about 3,000 mg/day, or about 2,000 mg/day to about 3,000 mg/day.
70. The composition according to any one of embodiments 29-69, wherein the pharmaceutical composition is administered in a single dose.
71. The composition according to any one of embodiments 29-69, wherein the pharmaceutical composition is administered in serial doses.
72. The composition according to any one of embodiments 29-71, wherein the therapeutic compound generates an immune response in an organism.
73. The composition according to any one of embodiments 29-71, wherein a toxoid is used to generate an immune response.
74. The composition according to embodiment 73, wherein the toxoid is a virus-like particle.
75. The composition according to embodiment 74, wherein the virus-like particle is a protein cage, a vault, or another such synthetic or biological structure.
76. The composition according to any one of embodiments 29-75, wherein a pharmaceutical composition including an immunopotentiator is employed to affect or improve the immune response.
77. The composition according to any one of embodiments 1-76, wherein, a component capable of coating an exposed surface covers at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% of the exposed surface of a scaffold, including, without limitation, a particulate lattice.
78. The composition according to any one of embodiments 1-77, wherein the transfer of an extract that is capable of coating exposed surfaces is to a protein, a protein cage, an antibody, a vault, a venom, a poison, a toxin, or a sub-cellular organelle.
79. The composition according to any one of embodiments 1-78, wherein a component capable of coating an exposed surface covers about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% of the exposed surface of a scaffold, including, without limitation, a particulate lattice.
80. A method to coat a surface, the method comprising the steps of a) producing, dissolving, or suspending a scaffold of an appropriate size and composition in a suitable solvent; b) loading one or more component in an extract that is capable of coating exposed surfaces onto the dissolved or suspended scaffold by any means, including but not limited to dialysis, diafiltration, tangential-flow filtration, spray-drying, supercritical-fluid evaporation, precipitation, or electroprecipitation; c) exposing the coated scaffold material to an appropriate-sized target particle in a manner which is sufficient to effect the transfer at least some of the component in an extract that is capable of coating exposed surfaces to the target surface; and, d) transferring at least some of the scaffold coating material onto the target surface.
81. A method for forming a scaffold and transferring to it one or more components capable of coating an exposed surface, the method comprising the steps of: a) choosing one or more a suitable component materials for the scaffold; b) choosing one or more suitable components for the component capable of coating an exposed surface; c) adding the components to a reaction mixture sequentially or else mixing the scaffold and coating components together all at once; d) rapidly dialyzing the mixture to cause the formation of the particulate scaffold; and, e) isolating or using the resultant particulate scaffold.
82. A method of treatment, wherein an individual is administered a pharmaceutical composition as defined in any one of embodiments 1-79 for the treatment of a disease, an infection, a cancer, a skin ailment, or other syndrome.
83. The method according to embodiment 82, wherein the disease is multiple sclerosis, rheumatoid arthritis, system lupus erthematosis.
84. The method according to embodiment 82, wherein the infection is a result of a bacterial, viral, or parasitic infection of an individual.
85. The method according to embodiment 82, wherein the cancer is Acute Lymphoblastic Leukemia; Acute Myeloid Leukemia; Karposi Sarcoma, Appendix Cancer; Adrenocortical Carcinoma; Anal Cancer; Basal Cell Carcinoma; Bladder Cancer; Blood Cancers Treatment, Brain Tumor, Adult; Brain Tumor, Brain Stem Glioma, Childhood; Brain Tumor, Cerebellar Astrocytoma, Childhood; Brain Tumor, Cerebral Astrocytoma, Childhood; Brain Tumor, Ependymoma, Childhood; Brain Tumor, Childhood (Other); Breast Cancer; Breast Cancer, Male; Carcinoid Tumor; Gastrointestinal, Carcinoma of Unknown Primary; Cervical Cancer; Colon Cancer; Endometrial Cancer; Esophageal Cancer; Extrahepatic Bile Duct Cancer; Ewings Family of Tumors (PNET); Extracranial Germ Cell Tumor, Childhood; Eye Cancer; Intraocular Melanoma; Gallbladder Cancer; Gastric Cancer (Stomach); Germ Cell Tumor; Extragonadal Gestational Trophoblastic Tumor; Head and Neck Cancer; Hypopharyngeal Cancer; Islet Cell Carcinoma; Kidney Cancer (renal cell cancer); Laryngeal Cancer; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic, Leukemia; Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoma; AIDS-Related Lymphoma; Central Nervous System (Primary); Lymphoma, Cutaneous T-Cell; Lymphoma, Hodgkin's Disease, Adult; Lymphoma, Hodgkin's Disease, Childhood; Lymphoma, Non-Hodgkin's Disease, Adult; Lymphoma, Non-Hodgkin's Disease, Childhood; Malignant Mesothelioma; Melanoma Merkel Cell Carcinoma; Metasatic Squamous Neck Cancer with Occult Primary; Multiple Myeloma and Other Plasma Cell Neoplasms; Mycosis Fungoides; Myelodysplastic Syndrome; Myeloproliferative Disorders; Nasopharyngeal Cancer; Neuroblastoma; Oral Cancer; Oropharyngeal Cancer; Osteosarcoma; Ovarian Epithelial Cancer; Ovarian Germ Cell Tumor; Pancreatic Cancer, Exocrine; Pancreatic Cancer, Islet Cell Carcinoma; Paranasal Sinus and Nasal Cavity Cancer; Parathyroid Cancer; Penile Cancer; Pituitary Cancer; Plasma Cell Neoplasm; Prostate Cancer; Rhabdomyosarcoma, Childhood; Rectal Cancer; Renal Cell Cancer (cancer of the kidney); Renal Pelvis and Ureter, Transitional Cell; Salivary Gland Cancer; Sezary Syndrome; Skin Cancer; Skin Cancer, Cutaneous T-Cell Lymphoma; Skin Cancer, Kaposi's Sarcoma; Skin Cancer, Melanoma; Small Intestine Cancer; Soft Tissue Sarcoma, Adult; Soft Tissue Sarcoma, Child; Stomach Cancer; Testicular Cancer; Thymoma, Malignant; Thyroid Cancer; Urethral Cancer; Uterine Cancer, Sarcoma Unusual Cancer of Childhood; Vaginal Cancer; Vulvar Cancer; or, Wilms' Tumor.
86. The method according to any one of embodiments 82-85, wherein the pharmaceutical composition is administered to an individual through an implantable piston, osmotic, or diffusion pumps, cannulae, diffusion-limiting gel, sponge or other device capable of administering the composition to an individual.

### EXAMPLES

The following non-limiting examples are provided for illustrative purposes only in order to facilitate a more complete understanding of the disclosed subject matter. These examples should not be construed to limit any of the embodiments described in the present specification, including those pertaining to the compositions, pharmaceutical compositions, or methods or uses disclosed herein.

### Example 1

The present invention provides a general vaccination method for living organisms, including plants, animals, and even microorganisms. In particular, a vaccine suitable for use in individuals, includes, without limitation, use in humans. A human vaccine is prepared by pre-assembling a particulate lattice by dialyzing a mixture of components from an aqueous resuspension of grape-seed extract (Vitaceae) sufficiently rapidly to cause the assembly of a particulate lattice with a surface loaded with proanthocyanidins, and then coating this lattice with proanthocyanidins obtained from grape-seed extract by any of a number of routes, such as the Konowalchuk (U.S. Patent Publication No. 20100221281) or Tempesta (U.S. Pat. No. 5,211,944) methods. Exposure of infectious agents, such as bacteria, viruses, fungi, or molds to the lattice-born proanthocyanidins results in the efficient transfer of the proanthocyanidin coating onto the infectious agent. The proanthocyanidin-coated infectious agent can then be safely administered to an individual, including a mammal, with or without killing the microorganism prior to such administration, to generate an immune response which is potentially attenuated by the accumulated coating material but still strong enough to prevent reinfection in subsequent challenges without undue adverse effects on the host. Through this exposure, the treated host plant or animal gains immunity to the microorganism without injury to itself.

### Example 2

Another aspect of the present invention provides a general method for coating synthetic particles with proanthocyanidins. A particulate lattice is pre-formed according to the procedure described in Example 1, and then a suspension of poly(styrene-divinyl benzene) particles of average diameter between 1 nm and 10 mm is added to the coated lattice suspension under appropriate conditions of temperature, pressure, and stirring, etc. After a sufficient transfer time, the proanthocyanidin-coated PS/DVB particles may be isolated from the reaction mixture, prepared for immediate use, or prepared for storage, or for any other such use. Such particulate lattices can be used to transfer coatings, including proanthocyanidin coatings, to a wide variety of microorganisms and other natural or synthetic particles.

### Example 3

Another aspect of the present invention provides a general method for coating microorganisms such as bacteria, viruses, molds, and fungi with proanthocyanidins obtained from another source, in this case the skin of blueberries (Ericaceae). In this example, an aqueous extract of blueberry skins is prepared according to any of a number of publically-available methods, and dextran beads with an average size in the range of 1 nm to 10 mm are suspended in the aqueous extract solution. To this mixture is added with stirring a water-miscible organic solvent in which the blueberry extract is insoluble and the mixture is stirred or otherwise agitated to effect the efficient transfer of the blueberry proanthocyanidins to the surface of the dextran beads. Once the transfer of the coating material is completed, a target microorganism, such as the human immunodeficiency virus (HIV), is added to the suspension of the proanthocyanidin-coated dextran beads and the materials are allowed to contact each other for a time which is sufficient to allow the transfer of the proanthocyanidin layer onto the virus. The coated virus is then used to immunize an appropriate animal by known methods, generating immunity in the host animal without a lethal immunological reaction.

### Example 4

Another aspect of the present invention provides a general method for the efficient coating of non-particulate surfaces, whether planar or three-dimensional, with a wide variety of coatings. As one example, a suitable particulate lattice is formed and loaded with a coating according to the procedure described in Example 1. The 3-D surface is mounted or suspended in an appropriate manner, and then the particulate mixture is exposed to the surface in the desired manner to optimize the transfer of the coating. The coating mixture is then removed, the target surface can be washed one or more times, and the resulting coated surface can be used for any of a large number of purposes.

### Example 5

Another aspect of the present invention provides a general method for the efficient preparation of a coated particulate lattice which can be used for any number of applications requiring the efficient transfer of the coating to a container surface. In this process, commercial grape-seed extract is suspended in water and prepared as per the Konowalchuk (U.S. Patent Publication No. 20100221281) method, except that after the filtration of the aqueous suspension poly(styrene)/divinylbenzene) (PS/DVB) particles of 100 nm diameter are added to the suspension. This aqueous suspension is then dialyzed against neat water using a 100,000 MWCO ultrafiltration membrane within a period of 24 hours. The retentate, containing the proanthocyanidin-coated PS/DVB particles, is then exposed to the inner surface of a poly(styrene) container for a time which is sufficient to transfer most or all of the proanthocyanidin coating to the poly(styrene) surface. Removal of the PS/DVB particles leaves the proanthocyanidin-coated poly(styrene) container, which can then be used for any of a number of research, clinical, manufacturing, or other purpose.

### Example 6

Another aspect of the present invention provides a general method for the efficient generation of therapeutic antibody candidates for a wide range of disease states, such as in this case for the treatment of human infection by the H1N1 virus. Proanthocyanidin-coated particles are prepared according to the process described in Example 1 and then exposed to an aqueous suspension of H1N1 virus particles for 30 minutes in order to coat the virus particles with proanthocyanidins. The proanthocyanidin-coated H1N1 virus particles are then administered to a mouse bearing a humanized immune system, such as a XenoMouse (Abgenix, Fremont, Calif.) or a Jax mouse (Jackson Laboratories, Bar Harbor, Calif.), using any of a number of methods well-known to those skilled in the art. The humanized mouse generates human antibodies to the proanthocyanidin-coated H1N1 virus particles, and these antibodies can be isolated by methods known to those skilled in the art to yield a set of human therapeutic antibodies which can then be developed further as therapeutic agents for safe and effective administration to a human.

### Example 7

Another aspect of the present invention provides a method for the vaccination of animals such as dogs. In this process, dextran particles of approximately 100 nm diameter are obtained and suspended in an aqueous environment. Proanthocyanidins prepared from blueberries are added to the dextran-particle suspension, and the mixture is dialyzed within 24 hours to yield proanthocyanidin-coated dextran particles. These coated particles are then exposed to a virus which can afflict dogs, such as canine parvovirus, for a period of time and under conditions suitable to effect efficient transfer of the proanthocyanidin coating to the virus. The proanthocyanidin-coated canine parvovirus is then injected into a dog under conditions which are suitable for the generation of an immune response, which in turn immunizes the dog against the canine parvovirus.

### Example 8

Another aspect of the present invention provides a general method for the generation of aptamers which are complementary to a particular toxin. In this process, dextran particles of approximately 100 nm diameter are obtained and suspended in an aqueous environment. Proanthocyanidins prepared from blueberries are added to the dextran-particle suspension, and the mixture is dialyzed within 24 hours to yield proanthocyanidin-coated dextran particles. These coated particles are then exposed to a virus, such as the human immunodeficiency virus (HIV) for a period of time and under conditions suitable to effect efficient transfer of the proanthocyanidin coating to the virus. The coated HIV toxoid is then used to generate a set of aptamers by methods which are well known to those skilled in the art. The aptamers are then isolated by any of several well-known means, such as high-performance liquid chromatography or gel electrophoresis, and then sequenced by any means well-known to those skilled in the art, such as by the use of an automated nucleic-acid sequencing instrument, to provide a set of nucleic acid sequences which can then be chemically or biologically synthesized using methods well-known to those skilled in the art. The anti-HIV aptamers can then be used for any number of purposes, such as administration to a human, plant, or animal to treat an infection, or tethering to a surface to be used to capture or detect HIV virus particles.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit.

At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A tannin-coated composition comprising a scaffold and one or more tannins, wherein the one or more tannin coats a surface of the scaffold.

2. The composition according to claim 1, wherein the one or more tannins are each naturally occurring or synthetically manufactured.

3. The composition according to claim 1, wherein the one or more tannins comprise one or more pseudotannins or one or more proanthocyanidins.

4. The composition according to claim 1, wherein the scaffold comprises a biological organism, a viral particle, a viron, a protein, a protein cage, a peptide, an antibody, a venom, a toxin, a vault, a nucleic acid, a fat, a lipid, a micelle, a liposome, a carbohydrate, a therapeutic compound, an active ingredient, a polymer latex, a metal object, a composite object, a fiber, a bead, and/or a rubber object.

5. The composition according to claim 1, wherein the scaffold comprises a carbohydrate support lattice or a carbohydrate particulate lattice.
